(19) United States
(12) Patent Application Publication
(10) Pub. No.: US 2024/0079104 A1
(43) Pub. Date: Mar. 7, 2024

(54) INFORMATION PROCESSING METHOD, INFORMATION PROCESSING APPARATUS, AND NONTRANSITORY COMPUTER-READABLE STORAGE MEDIUM

(71) Applicant: HASH PEAK K.K., Tokyo (JP)

(72) Inventor: Takuma MAEDA, Tokyo (JP)

(21) Appl. No.: 18/269,013

(22) PCT Filed: Dec. 22, 2021
(86) PCT No.: PCT/JP2021/047597

§ 371 Date: Jun. 22, 2023

(30) Foreign Application Priority Data
Dec. 28, 2020 (JP) 2020-219507

(51) Int. Cl. G16H 10/60; G06F 21/62; G06Q 20/36; G06Q 20/38

(52) U.S. Cl. CPC G16H 10/60; G06F 21/6245; G06Q 20/3678; G06Q 20/3825; G06Q 2220/00

(57) ABSTRACT

An information processing method is provided and includes: acquiring a patient ID for identifying a patient, the patient's attributes, and the patient's answers to medical questions; acquiring a doctor ID for identifying a doctor; transmitting the patient ID, the attributes, and the answers of the patient to a doctor terminal device corresponding to the acquired doctor ID; receiving an input of medical examination data for the patient through the doctor terminal device on which the patient ID, the attributes, and the answers of the patient are displayed; receiving a request to transmit medical data including the patient's attributes and answers and the input medical examination data; outputting a hash value of the medical data to a blockchain system; receiving a request for disclosure of the medical data; and outputting the medical data to a researcher terminal device when the patient's approval for the disclosure request from the researcher is obtained.

### CROSS REFERENCE TO RELATED APPLICATIONS

This application is the U.S. national phase under 35 U.S.C. § 371 of PCT International Application No. PCT/JP2021/047597 which has an International filing date of Dec. 22, 2021, designated the United States of America, and claims priority ton Patent Application No. 2020-219507 filed in Japan on Dec. 28, 2020. The entire disclosures of the above applications are hereby expressly incorporated by reference herein.

### BACKGROUND

### Technical Field

The present invention relates to an information processing method, an information processing apparatus, and a program.

### Related Art

Japanese Patent Laid-Open Publication No. 2020-064435 discloses a disease diagnosis, treatment, and prevention system that alerts medical personnel in charge when an abnormality is detected by using patient information, information of medical personnel in charge, and a plurality of pieces of inquiry information for disease diagnosis in which questions regarding adverse events in diseases and a plurality of answers thereto are combined.

However, the invention according to Japanese Patent Laid-Open Publication No. 2020-064435 has a problem that medical researchers cannot conduct medical research by using patient's medical data.

An object of one aspect is to provide an information processing method and the like capable of appropriately providing patient's medical data to medical researchers.

### SUMMARY

An information processing method according to one aspect includes: acquiring a patient ID for identifying a patient, the patient's attributes, and the patient's answers to medical questions; acquiring a doctor ID for identifying a doctor by causing a patient terminal device of the patient to read the doctor ID; transmitting the patient ID, the attributes, and the answers of the patient to a doctor terminal device corresponding to the acquired doctor ID; receiving an input of medical examination data for the patient by the doctor through the doctor terminal device on which the patient ID, the attributes, and the answers of the patient are displayed; receiving a request to transmit medical data including the patient's attributes and answers and the input medical examination data; outputting a hash value of the medical data to a blockchain system; receiving a request for disclosure of the medical data recorded in the blockchain system through a researcher terminal device of a researcher; and outputting the medical data to the researcher terminal device when the patient's approval for the disclosure request from the researcher is obtained.

According to one aspect, it becomes possible to appropriately provide patient's medical data to medical researchers.

### BRIEF DESCRIPTION OF THE DRAWINGS

The above and further objects and features will more fully be apparent from the following detailed description with accompanying drawings.

FIG. 1 is an explanatory diagram showing an overview of a medical data management system.

FIG. 2 is a block diagram showing a configuration example of a server.

FIG. 3 is an explanatory diagram showing an example of record layouts of a patient DB and a doctor DB.

FIG. 4 is an explanatory diagram showing an example of record layouts of a question DB and a medical data DB.

FIG. 5 is an explanatory diagram showing an example of record layouts of a researcher DB and a transaction history DB.

FIG. 6 is an explanatory diagram showing an example of a record layout of a management DB.

FIG. 7 is a block diagram showing a configuration example of a node of a blockchain.

FIG. 8 is a block diagram showing configuration examples of a doctor terminal and a patient terminal.

FIG. 9 is a block diagram showing a configuration example of a researcher terminal.

FIG. 10 is an explanatory diagram for explaining an operation of creating medical data with an electronic signature.

FIG. 11 is an explanatory diagram for explaining a process of recording medical data in a blockchain.

FIG. 12 is an explanatory diagram for explaining an operation of outputting medical data to a researcher terminal.

FIG. 13 is an explanatory diagram showing an example of an input screen for answers to questions.

FIG. 14 is an explanatory diagram showing an example of a screen for creating medical data.

FIG. 15 is an explanatory diagram showing an example of a medical data display screen on the patient terminal side.

FIG. 16 is an explanatory diagram showing an example of a medical data display screen on the patient terminal side.

FIG. 17 is an explanatory diagram showing an example of a medical data display screen on the patient terminal side.

FIG. 18 is an explanatory diagram showing an example of a disclosure request response screen on the patient terminal side.

FIG. 19 is an explanatory diagram showing an example of a medical data transaction history screen on the patient terminal side.

FIG. 20 is an explanatory diagram showing an example of a detailed medical data screen on the patient terminal side.

FIG. 21 is an explanatory diagram showing an example of a medical data management screen on the researcher terminal side.

FIG. 22 is an explanatory diagram showing an example of a transaction history screen on the researcher terminal side.

FIG. 23 is a flowchart showing a processing procedure when creating medical data.

FIG. 24 is a flowchart showing a processing procedure when creating medical data.

FIG. 25 is a flowchart showing a processing procedure when outputting medical data approved by a patient to a researcher terminal.

FIG. 26 is a flowchart showing a processing procedure when outputting medical data approved by a patient to a researcher terminal.

FIG. 27 is a flowchart showing a processing procedure when sharing medical data.

FIG. 28 is a flowchart showing a processing procedure when outputting information regarding research results to a patient terminal.

FIG. 29 is an explanatory diagram showing an example of a display screen for multiple times of medical data on the researcher terminal side.

FIG. 30 is an explanatory diagram showing an example of the record layout of a management DB in a fifth embodiment.

FIG. 31 is an explanatory diagram showing an example of a screen for receiving consent information.

FIG. 32 is a flowchart showing a processing procedure when outputting medical data re-approved by a patient to a researcher terminal.

### DETAILED DESCRIPTION

Hereinafter, the present invention will be described in detail with reference to the diagrams showing embodiments thereof.

### First Embodiment

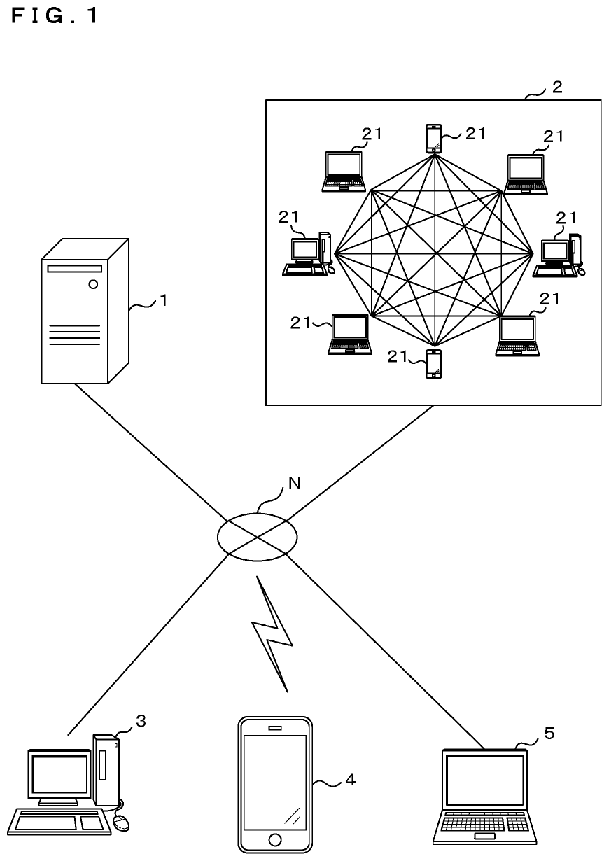

A first embodiment relates to a form in which medical data of a patient is provided to a researcher (medical researcher). FIG. 1 is an explanatory diagram showing an overview of a medical data management system. A system according to the present embodiment includes an information processing apparatus 1, a blockchain system 2, an information processing terminal 3, an information processing terminal 4, and an information processing terminal 5, and each apparatus transmits and receives information through a network N, such as the Internet.

The information processing apparatus 1 is an information processing apparatus that processes, stores, and transmits and receives various kinds of information. The information processing apparatus 1 is, for example, a server apparatus, a personal computer, or a general-purpose tablet PC (personal computer). In the present embodiment, the information processing apparatus 1 is assumed to be a server apparatus, and will be read as a server 1 below for the sake of simplicity.

The blockchain system 2 is a decentralized ledger technology or a decentralized network. The blockchain system 2 is configured by a plurality of nodes 21 that perform consensus processing. Each of the nodes 21 holds a copy of blockchain data through the execution of such consensus processing. The blockchain system 2 generates a unit of data called a block at predetermined intervals, and stores the data by linking the data like a chain.

The blockchain system 2 is autonomously managed through the use of a peer-to-peer network and a decentralized timestamp server. Since the data is stored in a chain form, once the data in the block is stored, it is difficult to retroactively change the data. The blockchain system 2 may be of public type, private type, or consortium type. The unit of data may be an individual transaction instead of a block. In addition, the data may be stored in a storage format, such as a directed acyclic graph, other than the chain form. Hereinafter, the blockchain system 2 will be read as a blockchain 2 for the sake of simplicity.

The information processing terminal 3 is a terminal device that creates medical data including patient's attributes, patient's answers to medical questions, and doctor's medical examination data for the patient and is used for electronic signature for medical data. The information processing terminal 3 is, for example, an information processing apparatus, such as a smart phone, a mobile phone, a tablet, a personal computer terminal and the like. Hereinafter, the information processing terminal 3 will be read as a doctor terminal 3 for the sake of simplicity.

The information processing terminal 4 is a terminal device that acquires patient's attributes and answers to questions, acquires medical data with an electronic signature, and accepts an input of approval information for a medical data disclosure request. The information processing terminal 4 is, for example, an information processing apparatus, such as a smart phone, a mobile phone, a wearable device such as Apple Watch (registered trademark), a tablet, a personal computer terminal and the like. Hereinafter, the information processing terminal 4 will be read as a patient terminal 4 for the sake of simplicity.

The information processing terminal 5 is a terminal device that receives and transmits a medical data disclosure request and receives medical data for which approval has been obtained for a disclosure request. The information processing terminal 5 is, for example, an information processing apparatus, such as a smart phone, a mobile phone, a tablet, a personal computer terminal and the like. Hereinafter, the information processing terminal 5 will be read as a researcher terminal 5 for the sake of simplicity.

The server 1 according to the present embodiment acquires a patient ID for identifying a patient, patient's attributes, and patient's answers to medical questions. The server 1 acquires a doctor ID by causing the patient terminal 4 to read the doctor ID for identifying a doctor. The server 1 transmits the patient ID, attributes, and answers of the patient to the doctor terminal 3 corresponding to the acquired doctor ID. The server 1 receives the doctor's input of medical examination data for the patient through the doctor terminal 3.

When a request for transmission of medical data including the attributes and answers of the patient and the input medical examination data is received from the doctor terminal 3, the server 1 calculates a hash value of the medical data. The server 1 outputs the calculated hash value of the medical data to the blockchain 2. When a request for disclosure of the medical data recorded in the blockchain 2 is received through the researcher terminal 5, the server 1 transmits the received disclosure request to the patient terminal 4 of the patient who provided the medical data. The server 1 transmits the medical data to the researcher terminal 5 when the patient's approval for the disclosure request is obtained.

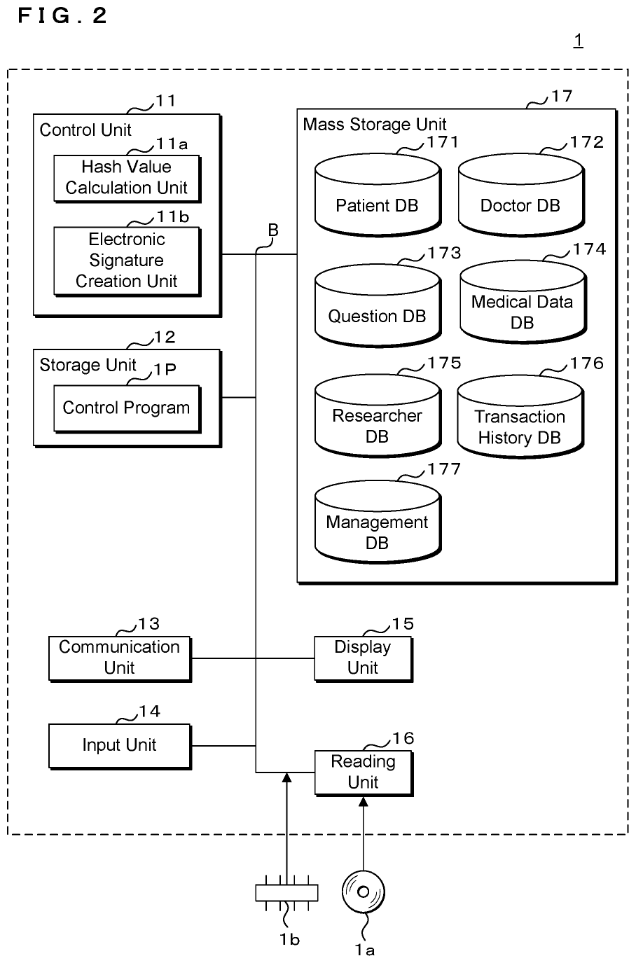

FIG. 2 is a block diagram showing a configuration example of the server 1. The server 1 includes a control unit 11, a storage unit 12, a communication unit 13, an input unit 14, a display unit 15, a reading unit 16, and a mass storage unit 17. The respective components are connected to each other by a bus B.

The control unit 11 includes an arithmetic processing device such as a CPU (Central Processing Unit), a MPU (Micro-Processing Unit), and a GPU (Graphics Processing Unit), and performs various kinds of information processing, control processing, and the like related to the server 1 by reading and executing a control program 1P (program product) stored in the storage unit 12. In addition, examples of the control program 1P include programs for generation and execution of a smart contract such as Geth (Go-Ethereum) of Ethereum, remittance of virtual currency, account creation, mining, and the like.

In addition, the control unit 11 includes a hash value calculation unit 11a and an electronic signature creation unit 11b. The hash value calculation unit 11a performs processing for calculating the hash values of various kinds of information by using a cryptographic hash function. The electronic signature creation unit 11b creates a signature for digital authentication based on public key cryptography in order to prevent counterfeiting or tampering. In addition, although the control unit 11 is described as a single processor in FIG. 2, the control unit 11 may be a multiprocessor.

The storage unit 12 includes memory elements such as a RAM (Random Access Memory), a ROM (Read Only Memory), and stores the control program 1P, data, and the like necessary for the control unit 11 to perform processing. In addition, the storage unit 12 temporarily stores data and the like necessary for the control unit 11 to perform arithmetic processing. The communication unit 13 is a communication module for performing processing related to communication, and transmits and receives information to and from the node 21 of the blockchain 2, the doctor terminal 3, the patient terminal 4, the researcher terminal 5, and the like through the network N.

The input unit 14 is an input device such as a mouse, a keyboard, a touch panel, or a button, and outputs received operation information to the control unit 11. The display unit 15 is a liquid crystal display, an organic EL (electroluminescence) display, or the like, and displays various kinds of information according to instructions from the control unit 11.

The reading unit 16 reads a portable storage medium 1a including CD (Compact Disc)-ROM or DVD (Digital Versatile Disc)-ROM. The control unit 11 may read the control program 1P from the portable storage medium 1a through the reading unit 16 and store the control program 1P in the mass storage unit 17. In addition, the control unit 11 may download the control program 1P from another computer through the network N or the like and store the control program 1P in the mass storage unit 17. In addition, the control unit 11 may read the control program 1P from a semiconductor memory 1b.

Examples of the mass storage unit 17 include recording media such as an HDD (Hard disk drive), an SSD (Solid State Drive). The mass storage unit 17 includes a patient DB (database) 171, a doctor DB 172, a question DB 173, a medical data DB 174, a researcher DB 175, a transaction history DB 176, and a management DB 177.

The patient DB 171 stores the attributes of patients. The doctor DB 172 stores the attributes of doctors. The question DB 173 stores patient answers to medical questions. The medical data DB 174 stores patient's medical data. The researcher DB 175 stores the attributes of researchers. The transaction history DB 176 stores the transaction history of medical data. The management DB 177 stores management information of medical data.

In addition, in the present embodiment, the storage unit 12 and the mass storage unit 17 may be configured as an integrated storage device. In addition, the mass storage unit 17 may be configured by a plurality of storage devices. In addition, the mass storage unit 17 may be an external storage device connected to the server 1.

In addition, in the present embodiment, the server 1 is described as a single information processing apparatus. However, the processing may be performed in a decentralized manner by a plurality of information processing apparatuses, or the server 1 may be configured by a virtual machine.

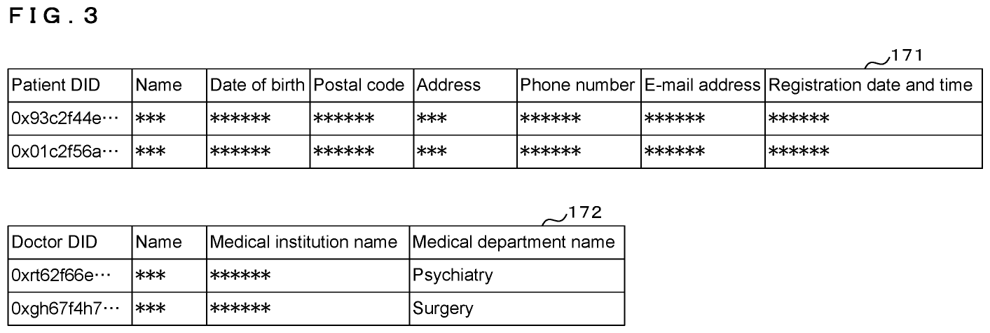

FIG. 3 is an explanatory diagram showing an example of record layouts of the patient DB 171 and the doctor DB 172. The patient DB 171 includes a patient DID (Decentralized Identifier) column, a name column, a date of birth column, a postal code column, an address column, a phone number column, an e-mail address column, and a registration date and time column. The patient DID column stores uniquely identified patient DIDs in order to identify each patient. The DID is a decentralized identity (decentralized ID) in which necessary information of user's attribute information held by each data owner is linked within the scope permitted by the user after the user secures the control right regarding the user's attribute information. The DID realizes a unique identifier that does not overlap by using a decentralized system constructed by the blockchain 2, so that it is possible to maintain immutability or high reliability against external viewing and tampering. In addition, although an example in which the patient ID is a patient DID has been described in the present embodiment, the present invention is not limited thereto. The patient ID may be a conventional (existing) ID for identifying a patient, biometric information using a face or fingerprint, and the like.

The name column stores the names of patients. The date of birth column stores the dates of birth of patients. The postal code column stores postal codes based on the current addresses of patients. The address column stores the current addresses of patients. The phone number column stores the phone numbers of patients. The e-mail address column stores the e-mail addresses of patients. The registration date and time column stores date and time information when patient's attributes are registered.

The doctor DB 172 includes a doctor DID column, a name column, a medical institution name column, and a medical department name column. The doctor DID column stores uniquely identified doctor DIDs in order to identify each doctor. In addition, although an example in which the doctor ID is a doctor DID has been described in the present embodiment, the present invention is not limited thereto. The doctor ID may be a conventional ID for identifying a doctor, biometric information using a face or a fingerprint, and the like. The name column stores the names of doctors. The medical institution name column stores the names of the medical institution where each doctor works. The medical department name column stores the names of medical departments, such as psychiatry, dermatology, and surgery.

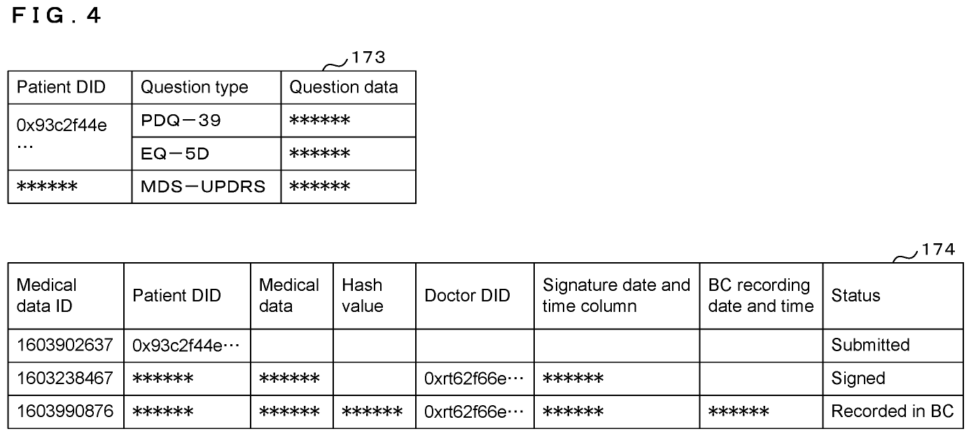

FIG. 4 is an explanatory diagram showing an example of the record layout of the question DB 173 and the medical data DB 174.

The question DB 173 includes a patient DID column, a question type column, and a question data column. The patient DID column stores patient DIDs for identifying patients. The question type column stores the types of medical questions. Examples of the question type include PDQ (Parkinson's Disease Questionnaire)-39, MDS-UPDRS (Movement Disorder Society-sponsored revision of the Unified Parkinson's Disease Rating Scale), and EQ-5D (EuroQOL 5 Dimensions). In addition, the types of questions described above are examples, and are not limited thereto. The question data column stores medical questions and patient answers to each question.

The medical data DB 174 includes a medical data ID column, a patient DID column, a medical data column, a hash value column, a doctor DID column, a signature date and time column, a BC recording date and time column, and a status column. The medical data ID column stores uniquely identified medical data IDs in order to identify each piece of medical data. The patient DID column stores patient DIDs for identifying patients.

The medical data column stores patient's medical data in association with the medical data ID. In the medical data column, medical data including patient's attributes, patient's answers to medical questions, and doctor's medical examination data for patients are stored. Examples of the medical data include a JSON (JavaScript (registered trademark) Object Notation) format file and an XML (Extensible Markup Language) format file. The hash value column stores hash values of medical data.

The doctor DID column stores doctor DIDs for identifying doctors. The signature date and time column stores date and time information when the medical data was electronically signed. The BC recording date and time column stores the date and time when the medical data was recorded in the blockchain 2. The status column stores the status of medical data. Examples of the status of medical data include “draft saved”, “submitted”, “signed” or “recorded in BC”. The “draft saved” status is a status in which patient's attributes and patient's answers to questions are saved as drafts. The “submitted” status is a status indicating that patient's attributes and patient's answers to questions have been submitted (transmitted) to the server 1. The “signed” status is a status indicating that medical data including patient's attributes, patient's answers to medical questions, and doctor's medical examination data for the patient has been signed. The “recorded in BC” status is a status indicating that the medical data has been recorded on the blockchain 2.

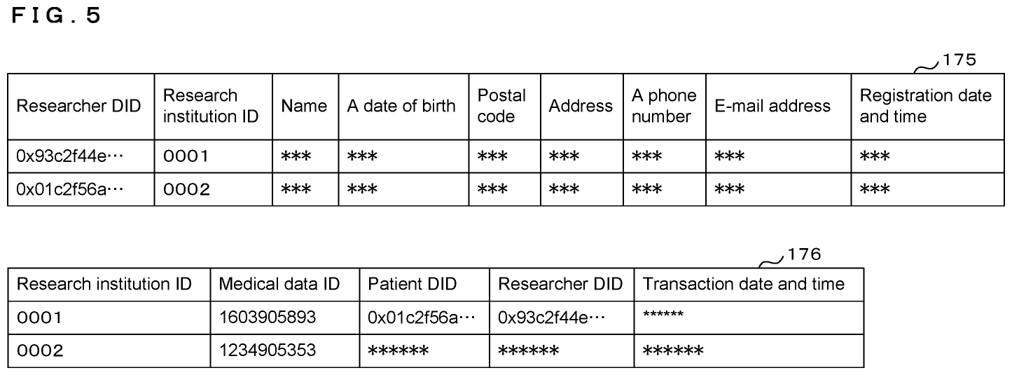

FIG. 5 is an explanatory diagram showing an example of the record layout of the researcher DB 175 and the transaction history DB 176. The researcher DB 175 includes a researcher DID column, a research institution ID column, a name column, a date of birth column, a postal code column, an address column, a phone number column, an e-mail address column, and a registration date and time column. The researcher DID column stores uniquely identified researcher DIDs in order to identify each researcher. In addition, although an example in which the researcher ID is a researcher DID has been described in the present embodiment, the present invention is not limited thereto. The researcher ID may be a conventional ID for identifying a researcher, biometric information using a face or a fingerprint, and the like.

The research institution ID column stores research institution IDs for identifying research institutions to which researchers belong. The research institution is an organization (institution) or its facility for conducting research, testing, appraisal, or the like. The name column stores the names of researchers. The date of birth column stores the dates of birth of researchers. The postal code column stores postal codes based on the current addresses of researchers. The address column stores the current addresses of researchers. The phone number column stores the phone numbers of researchers. The e-mail address column stores the e-mail addresses of researchers. The registration date and time column stores date and time information when researcher's attributes are registered.

The transaction history DB 176 includes a research institution ID column, a medical data ID column, a patient DID column, a researcher DID column, and a transaction date and time column. The research institution ID column stores research institution IDs for identifying research institutions. The medical data ID column stores medical data IDs for identifying medical data. The patient DID column stores patient DIDs for identifying patients. The researcher DID column stores researcher DIDs for identifying researchers. The transaction date and time column stores the dates and times of transactions of medical data between patients and researchers.

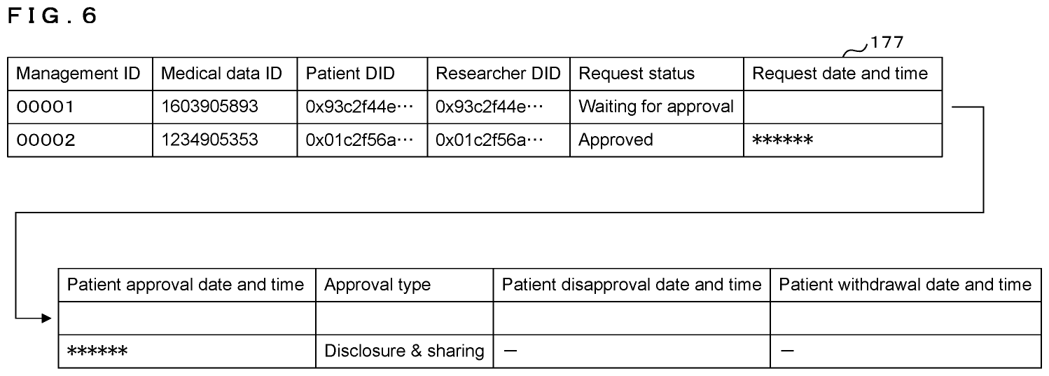

FIG. 6 is an explanatory diagram showing an example of the record layout of the management DB 177. The management DB 177 includes a management ID column, a medical data ID column, a patient DID column, a researcher DID column, a request status column, a request date and time column, a patient approval date and time column, an approval type column, a patient disapproval date and time column, and a patient withdrawal date and time column. The management ID column stores uniquely identified management data IDs in order to identify each piece of management data. The medical data ID column stores medical data IDs for identifying medical data. The patient DID column stores patient DIDs for identifying patients. The researcher DID column stores researcher DIDs for identifying researchers.

The request status column stores a patient's answer status for a request for disclosure of medical data. In the request status column, “data not requested”, “waiting for approval”, “approved”, “withdrawn”, “pending”, and the like are stored. The request date and time column stores date and time information when a medical data disclosure request is received. The patient approval date and time column stores date and time information when the patient approved the use of medical data.

The approval type column stores an approval type for medical data. As the approval type, there are a first approval for a medical data disclosure request and an approval including the first approval and a second approval of a patient for a sharing request to share the medical data, for which the first approval for the medical data disclosure request has been obtained, with other researchers. Hereinafter, for the sake of simplicity, the first approval is read as “disclosure only”, and the approval including the first approval and the second approval is read as “disclosure & sharing”. The patient disapproval date and time column stores date and time information when the patient disapproved the use of medical data. The patient withdrawal date and time column stores date and time information when the patient withdrew the approval for approved medical data.

In addition, the storage form of each DB described above is an example, and other storage forms may be used as long as the relationship between the pieces of data is maintained.

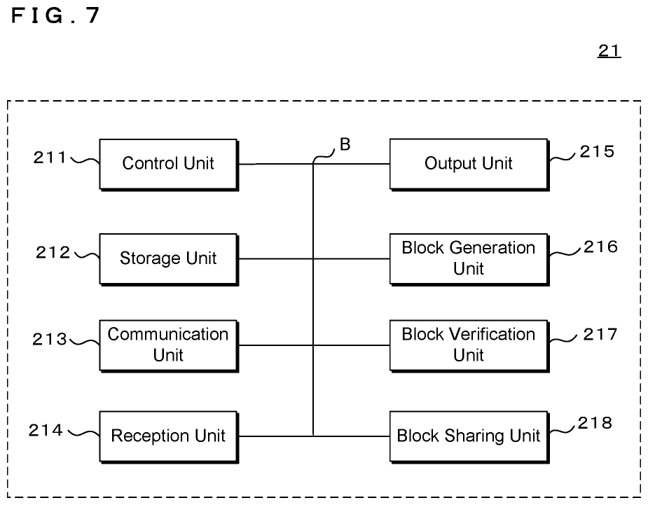

FIG. 7 is a block diagram showing a configuration example of the node 21 of the blockchain 2. The node 21 of the blockchain 2 includes a control unit 211, a storage unit 212, a communication unit 213, a reception unit 214, an output unit 215, a block generation unit 216, a block verification unit 217, and a block sharing unit 218. The respective components are connected to each other by the bus B.

The control unit 211 always holds the latest blockchain (ledger: copy of blockchain data) in the storage unit 212 in autonomous decentralized cooperation with control units of the other nodes 21 (terminals). The storage unit 212 stores a blockchain (ledger) including transactions broadcasted to the decentralized network, information necessary for verification processing of information in the block, and the like.

The communication unit 213 is a communication module for performing processing related to communication. The reception unit 214 receives information to be recorded in a decentralized network, which is a blockchain managed by the blockchain 2, from an external node. The output unit 215 outputs information of the blockchain held by itself in response to a request from the external node.

The block generation unit 216 generates a block to be added to the blockchain based on the information received by the reception unit 214. The block generation unit 216 generates a block including information based on the previous block and the information received by the reception unit 214. In addition, the block generation unit 216 performs, for example, processing for searching for a nonce or processing for adding a signature, as predetermined consensus processing, on a block generated by itself or a block generated by the node 21 of another blockchain through the block sharing unit 218 described later, and then adds the block to the blockchain (ledger) managed by itself. In addition, a block obtained by performing predetermined consensus processing on the block, which is generated by the block generation unit 216, by a plurality of nodes 21 is the block finally added to the blockchain 2.

The block verification unit 217 verifies information in a block when adding the block to the blockchain held by itself. Normally, the block to be added is a block that satisfies the rule earliest in the node 21 group including its own node. However, considering a case where the malicious node 21 is included and the like, it may be verified whether or not the rule is actually satisfied.

The block sharing unit 218 transmits and receives information to and from the node 21 belonging to the blockchain 2. More specifically, the block sharing unit 218 appropriately transmits the information received by the reception unit 214, the block generated by the block generation unit 216, blocks received from other nodes 21, and the like to another node 21. Therefore, these pieces of information and the latest blockchain 2 are shared by all the nodes 21 as much as possible.

In addition, the configuration in FIG. 7 is merely an example, and the node 21 of the blockchain can have any configuration as long as it is possible to perform predetermined consensus processing for a plurality of nodes to share and manage the blockchain 2 that is difficult to tamper with and it is possible to add information to the decentralized network and refer to information recorded in the decentralized network in response to a request from an external node.

In addition, the server 1 may be the node 21 of the blockchain 2.

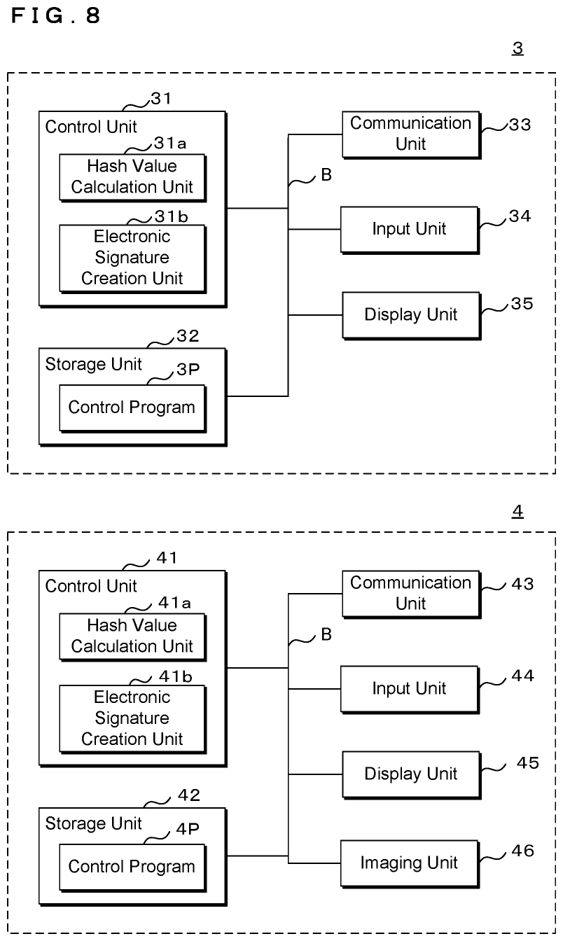

FIG. 8 is a block diagram showing configuration examples of the doctor terminal 3 and the patient terminal 4. The doctor terminal 3 includes a control unit 31, a storage unit 32, a communication unit 33, an input unit 34, and a display unit 35. The respective components are connected to each other by the bus B.

The control unit 31 includes an arithmetic processing unit such as a CPU and an MPU, and performs various kinds of information processing, control processing, and the like related to the doctor terminal 3 by reading and executing a control program 3P (program product) stored in the storage unit 32. In addition, the control unit 31 includes a hash value calculation unit 31a and an electronic signature creation unit 31b. The hash value calculation unit 31a performs processing for calculating a wallet address and the like by using a cryptographic hash function. In addition, examples of the control program 3P include programs for generation and execution of a smart contract such as Geth of Ethereum, remittance of virtual currency, account creation, mining, and the like. The electronic signature creation unit 31b creates a signature for digital authentication based on public key cryptography in order to prevent counterfeiting or tampering.

In addition, although the control unit 31 is described as a single processor in FIG. 8, the control unit 31 may be a multiprocessor. The storage unit 32 includes memory elements such as a RAM and a ROM, and stores the control program 3P, data, and the like necessary for the control unit 31 to perform processing. In addition, the storage unit 32 temporarily stores data and the like necessary for the control unit 31 to perform arithmetic processing. The communication unit 33 is a communication module for performing processing related to communication, and transmits and receives information to and from the server 1 and the like through the network N. The input unit 34 may be a keyboard, a mouse, or a touch panel integrated with the display unit 35. The display unit 35 is a liquid crystal display, an organic EL display, or the like, and displays various kinds of information according to instructions from the control unit 31.

The patient terminal 4 includes a control unit 41, a storage unit 42, a communication unit 43, an input unit 44, a display unit 45, and an imaging unit 46. The respective components are connected to each other by the bus B. In addition, since the control unit 41, the storage unit 42, the communication unit 43, the input unit 44, and the display unit 45 of the patient terminal 4 are the same as the control unit 31, the storage unit 32, the communication unit 33, the input unit 34, and the display unit 35 of the doctor terminal 3, the description thereof will be omitted. The imaging unit 46 is an imaging device, such as a CCD (Charge Coupled Device) camera and a CMOS (Complementary Metal Oxide Semiconductor) camera. In addition, the imaging unit 46 may be directly and externally connected to the patient terminal 4 for imaging without being built in the patient terminal 4.

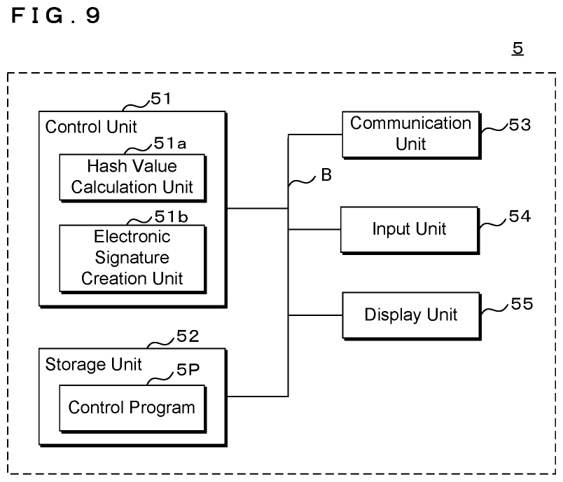

FIG. 9 is a block diagram showing a configuration example of the researcher terminal 5. The researcher terminal 5 includes a control unit 51, a storage unit 52, a communication unit 53, an input unit 54, and a display unit 55. The respective components are connected to each other by the bus B. In addition, since the control unit 51, the storage unit 52, the communication unit 53, the input unit 54, and the display unit 55 of the researcher terminal 5 are the same as the control unit 31, the storage unit 32, the communication unit 33, the input unit 34, and the display unit 35 of the doctor terminal 3, the description thereof will be omitted.

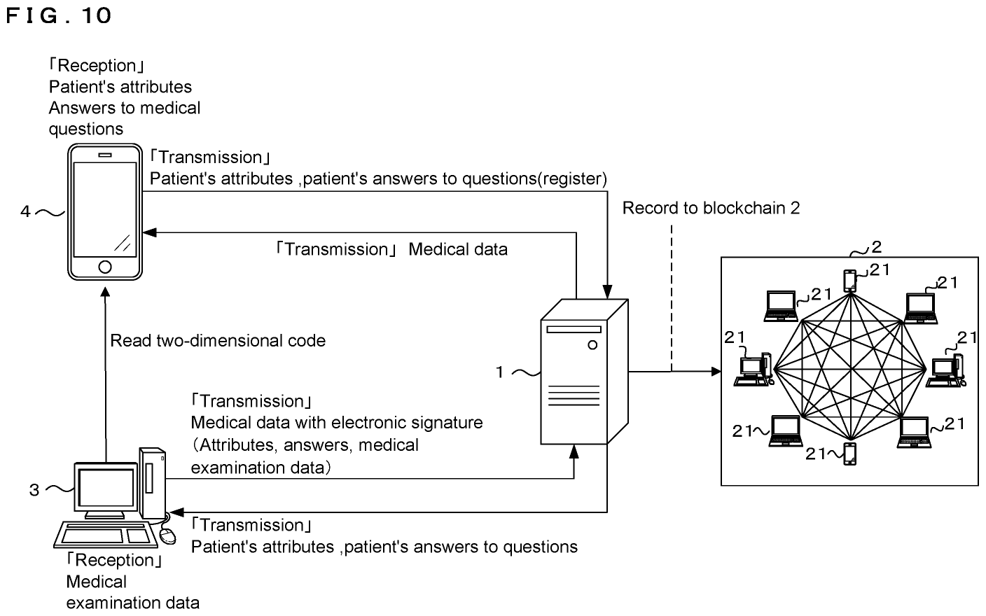

FIG. 10 is an explanatory diagram for explaining an operation of creating medical data with an electronic signature. First, processing for registering patient's attributes in advance will be described. Patient's attributes include patient's name, sex, age, date of birth, zip code, address, phone number, e-mail address, medical history, medication history, blood type, and the like. In addition, patient's attributes may include biometric data (for example, blood pressure, electrocardiogram, heart sound, or X-ray absorptivity), which are various physiological and anatomical information emitted by the living body.

Specifically, the patient terminal 4 receives an input of patient's attributes and transmits the received patient's attributes to the server 1. The server 1 receives the patient's attributes transmitted from the patient terminal 4 and issues a patient DID. For example, the server 1 issues a patient DID for identifying a patient by using a platform such as ERC725/735 of Ethereum (registered trademark) or Sovrin Network.

The server 1 stores the received patient's attributes in the patient DB 171 in association with the issued patient DID. Specifically, the server 1 stores the patient's name, date of birth, zip code, address, phone number, e-mail address, and registration date and time in the patient DB 171 as one record in association with the patient DID.

Next, processing for registering patient's answers to medical questions in advance will be described. For medical questions, for example, in Parkinson's disease, PDQ-39, which is a disease-specific QOL (Quality of Life) questionnaire for a person with Parkinson's disease, may be used. Specifically, the patient terminal 4 receives the patient's selection of question type (for example, PDQ-39, MDS-UPDRS, or EQ-5D). According to the received question type, the patient terminal 4 acquires the corresponding questions from the storage unit or the external device and displays the questions on the screen.

The patient terminal 4 receives the patient's answers to the questions displayed on the screen, transmits the received answers to the server 1 in association with the patient DID. The server 1 receives the patient DID and the answers to the questions transmitted from the patient terminal 4, and stores the received patient DID and answers in the question DB 173. Specifically, the server 1 stores question data including the question type, questions, and answers to the questions, as one record, in the question DB 173 so as to be associated with the received patient DID.

Moreover, in addition to the patient's attributes and the patient's answers to questions, vital data (biometric data) measured by a wearable device that enables measurement of blood pressure, heart rate, respiratory rate, body temperature, and the like or a terminal including a vital sensor may be registered in the server 1. For example, the patient terminal 4 acquires vital data including blood pressure, heart rate, respiration rate, body temperature, and the like through a wearable device. The patient terminal 4 registers the acquired vital data in the server 1 together with the patient's attributes and the patient's answers to questions.

Next, processing for creating medical data with an electronic signature will be described. The medical data includes patient's attributes, patient's answers to medical questions, and doctor's medical examination data for the patient.

The doctor terminal 3 of the doctor generates a code describing the doctor DID for identifying the doctor by using a library for generating the code. Examples of the code include a one-dimensional code or a two-dimensional code. The one-dimensional code may be, for example, a barcode that represents numerical values or characters by the thickness of striped lines. The two-dimensional code may be, for example, a QR code (registered trademark) based on a display method having information in the horizontal and vertical directions, as opposed to the one-dimensional code having information only in the horizontal direction. An example of the two-dimensional code will be described below. A doctor DID may be issued by using a platform such as Ethereum's ERC725/735 or the Sovrin Network, for example.

The doctor terminal 3 displays the generated two-dimensional code on the screen. When the patient is diagnosed by the doctor, the patient terminal 4 reads the two-dimensional code displayed on the doctor terminal 3. The patient terminal 4 acquires the doctor DID from the read two-dimensional code by using a two-dimensional code analysis library. In addition, although the example in which a two-dimensional code is used has been mentioned in the present embodiment, the present invention is not limited thereto. For example, a doctor DID stored in an IC tag (Integrated Circuit Tag), an RF tag (Radio Frequency Tag), an RFID (Radio Frequency Identifier) chip (microchip), or the like may be read by an RF reader, or a doctor DID stored in a barcode may be read. Furthermore, a doctor DID may be received by manual input.

The patient terminal 4 transmits the acquired doctor DID and patient DID to the server 1. The server 1 receives the doctor DID and the patient DID transmitted from the patient terminal 4. The server 1 acquires the patient's attributes and answers to questions based on the received patient DID. Specifically, the server 1 acquires the patient's attributes from the patient DB 171 based on the patient DID. The server 1 acquires the answers to questions from the question DB 173 based on the patient DID.

The server 1 transmits the patient DID and the acquired patient's attributes and answers to questions to the doctor terminal 3 corresponding to the received doctor DID. The doctor terminal 3 receives the patient DID, the patient's attributes, and the answers to questions transmitted from the server 1. The doctor terminal 3 receives the doctor's input of medical examination data for the patient. For example, the medical examination data includes severity classification of diseases such as Parkinson's disease, prescription information (ingredient names of drug, dosage form, dosage, and the like), blood pressure, and pulse rate.

When an instruction to electronically sign the medical data including the patient's attributes, the answers to questions, and the medical examination data is received, the doctor terminal 3 performs electronic signature processing (encryption) on the medical data. The electronic signature processing is a function of applying an electronic signature to given data using a private key. Normally, a hash value obtained by converting the given data with a cryptographic hash function is encrypted. The cryptographic hash function is a one-way cryptographic method for detecting the tampering of a digital document, and the output value is always fixed to 64 digits as hexadecimal notation. Any hash function can be used. For example, SHA (Secure Hash Algorithm)-1 or SHA-256 can be used. In addition, when the size of data to be electronically signed is small, the encrypted data itself may be used as the electronic signature instead of the hash value.

In addition, although the doctor electronically signs the medical data in the present embodiment, but the present invention is not limited thereto. For example, medical data may be electronically signed by a medically qualified person such as a clinical laboratory technician, a physical therapist, or a caregiver.

The doctor terminal 3 transmits the medical data with an electronic signature to the server 1 in association with the patient DID and the doctor DID. The server 1 receives the patient DID, the doctor DID, and the medical data with an electronic signature transmitted from the doctor terminal 3. The server 1 decrypts the medical data with an electronic signature by using the public key. The server 1 creates a medical data ID for identifying the medical data. For example, the server 1 may create a non-fungible token (NFT) that can prove “value”, as a medical data ID, by using ERC721, which is one smart contract standard of Ethereum. If the ERC721 standard is used, all medical data IDs on the blockchain 2 are disclosed, but it is possible to add owner or attribution information because tampering is not possible.

The server 1 stores the patient DID, the decrypted medical data, the doctor DID, the date and time of the electronic signature, and the “signed” status, as one record, in the medical data DB 174 in association with the created medical data ID. The server 1 transmits the medical data to the patient terminal 4. The patient terminal 4 receives the medical data transmitted from the server 1 and displays the medical data on the screen. When an instruction to output (record) the medical data to the blockchain 2 is received from the patient, the patient terminal 4 transmits the instruction for output to the blockchain 2 to the server 1. The output instruction includes the patient DID and the medical data ID. The server 1 receives the instruction for output to the blockchain 2 transmitted from the patient terminal 4, and outputs a hash value of the medical data to the blockchain 2 according to the received output instruction.

Specifically, the server 1 calculates the hash value of the medical data by using a cryptographic hash function. For example, the server 1 calculates the hash value of the medical data by adopting a hash function using the SHA2-256 algorithm. In addition, other encryption methods may be used instead of the hash function. The server 1 transmits the calculated hash value of the medical data to any node 21 of the blockchain 2 in association with the patient DID, the doctor DID, and the medical data ID. The node 21 of the blockchain 2 receives and records the patient DID, the doctor DID, the medical data ID, and the hash value of the medical data transmitted from the server 1. The server 1 stores in the medical data DB 174 the hash value of the medical data, the date and time of recording to the blockchain 2, and the status of “recorded in BC” in association with the medical data ID.

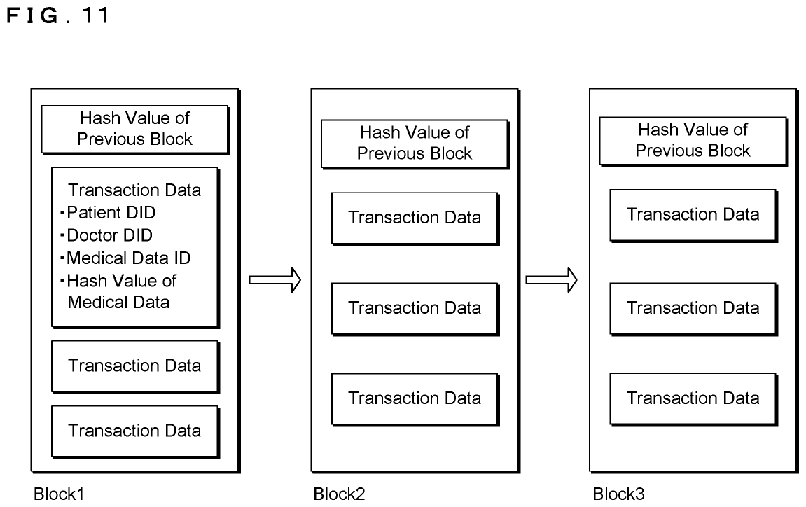

FIG. 11 is an explanatory diagram for explaining the process of recording medical data in the blockchain 2. One node 21 of the blockchain 2 receives transaction data (transaction) transmitted from the server 1. As shown in the diagram, the transaction data includes a patient DID, a doctor DID, a medical data ID, and a hash value of the medical data. The node 21 generates a block including at least previous block management information created based on information of the blockchain managed by itself and the patient DID, the doctor DID, the medical data ID, and the hash value of the medical data included in the received transaction data.

Then, each node 21 of the blockchain 2 performs processing according to a predetermined consensus algorithm. For example, when blockchain technology related to Bitcoin (registered trademark) is applied to this system, the node 21, the node 21, the node 21, . . . perform arithmetic processing called PoW (Proof of Work) and determine the node 21 to update the blockchain 2. PoW is arithmetic processing (mining) for searching for a correct value from the hash value stored in advance in a block immediately before a newly generated block.

When generating a new block, the node 21 generates a hash value obtained by hashing the transaction data and the hash value stored in a block immediately before the newly generated block, and stores the hash value in the latest block. For example, as shown in FIG. 11, when generating a block 3, the node 21 generates a hash value by hashing the hash value stored in a block 2 and a plurality of pieces of transaction data, and stores the hash value in the block 3. For example, in the case of bitcoin, the node 21 generates a hash value by inserting a nonce value into the header in addition to the transaction data, in order to increase the difficulty of arithmetic processing. The nonce value is a value in which a predetermined number of zeros are consecutive in the header of the hash value, and the processing load of arithmetic processing is adjusted by designing the number of zeros related to the nonce value to an appropriate number.

When generating the latest block, each node 21 solves a search problem of searching for a correct value (nonce value in the example of Bitcoin) from the hash values included in the previous block. For example, as shown in FIG. 11, when generating a new block 3, each node 21 searches for a correct value from the hash values stored in the blocks of the block 2. The probability of finding the correct value in one arithmetic processing is low, and for example, when applying blockchain technology related to virtual currency such as bitcoin, it takes about 10 minutes on average. The respective nodes 21 simultaneously perform arithmetic processing of the search problem to search for the correct value. Then, the node 21 that finds the correct value earliest is given the authority to generate the latest block. When a fraudster attempts to rewrite the data of the blockchain, it is necessary to generate blocks at a speed exceeding the arithmetic processing speed of all bona fide verifiers (node 21). Therefore, it is extremely difficult to actually perform unauthorized rewriting.

When the search is successful, the node 21 generates a latest block including the verified transaction data (the patient DID, the doctor DID, the medical data ID, and the hash value of the medical data), and notifies the other nodes 21, 21, 21, . . . of data related to the block. In this case, as described above, the node 21 generates a hash value from the hash value and the transaction data stored in a block immediately before a newly generated block, stores the hash value in the new block, and then provides notification to each node 21.

For example, as shown in FIG. 11, the node 21 generates a hash value by hashing the transaction data and the hash value stored in the block 2, stores the hash value in the new block 3, and provides notification to the other nodes 21, 21, 21, . . . . Since the hash values stored in the new block include the hash value of the block immediately before the new block, the blocks that make up the blockchain are associated with each other in chronological order. The other nodes 21, 21, 21, . . . store the transaction data in the storage unit 212 after checking the validity of the notified block data. As a result, data related to the same blockchain is duplicated and stored in the storage unit 212 of each of the node 21, 21, 21, . . . .

In addition, the administrator (minor) of the node 21 that has succeeded in the search is given a reward for the successful search. That is, the administrator of the node 21 that has succeeded in the search is paid a commission for each transaction stored in the block. As a result, the administrator is given an incentive to input a computational resource called the node 21.

In addition, PoW is used as a method of determining the blockchain update authority in the above, but the present embodiment is not limited thereto. For example, POI (Proof of Importance), POS (Proof of Stake) and the like may be used. In addition, although the system to which the Bitcoin technology is applied has been described above, the present embodiment is not limited thereto. For example, in addition to Bitcoin, blockchain technology related to Ethereum, Hyperledger Fabric, and the like may be applied.

Next, a process of outputting medical data to the researcher terminal 5 in response to a medical data disclosure request from a researcher who conducts medical research using the medical data recorded in the blockchain 2 will be described.

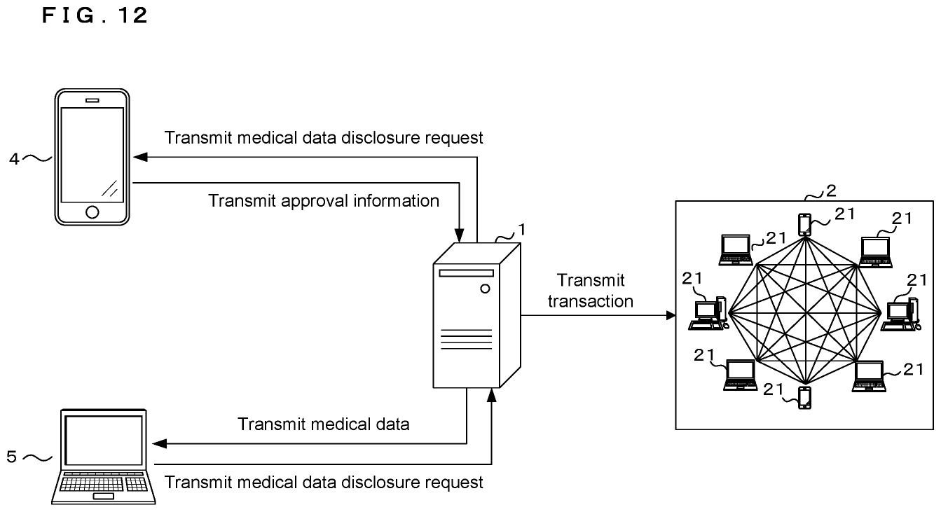

FIG. 12 is an explanatory diagram for explaining the operation of outputting medical data to the researcher terminal 5. In addition, researcher's attributes are registered in advance. The researcher's attributes include the researcher's name, date of birth, zip code, address, phone number, e-mail address, profile, research purpose, and the like. In addition, since the researcher registration processing is the same as the patient registration processing, the description thereof will be omitted.

When a medical data disclosure request is received from a researcher, the researcher terminal 5 transmits the received disclosure request to the server 1 in association with the researcher DID and the medical data ID. The server 1 transmits the researcher DID, the medical data ID, and the disclosure request transmitted from the researcher terminal 5 to the patient terminal 4 of the patient who provided the medical data. In addition, the researcher's message or profile may be transmitted to the patient terminal 4 together with the disclosure request. In this case, the patient terminal 4 displays the researcher's message or profile on the screen together with the disclosure request.

When approval information for the medical data disclosure request is received in response to the disclosure request transmitted from the server 1, the patient terminal 4 transmits the received approval information to the server 1. The server 1 creates a transaction for paying a predetermined token from the researcher to the patient according to the approval information transmitted from the patient terminal 4. The transaction is a transaction record in the blockchain 2, and stores various kinds of information and transfer of value between participants in the blockchain 2. The created transaction includes the researcher's wallet address, the patient's wallet address, the amount of tokens paid, and the date and time of transaction.

The wallet address is a virtual currency account number created for each transaction, and is displayed as a character string or QR code. The wallet address is an address derived mathematically based on the public key cryptography used in the blockchain 2. In addition, the wallet addresses of the transmission source and the transmission destination are recorded in the node 21 of the blockchain 2, and are not tampered with as long as the system of the blockchain 2 continues to maintain the wallet addresses. Since the wallet address is computationally generated, it is possible to create the wallet address online or offline.

The server 1 transmits the created transaction to any node 21 of the blockchain 2. The node 21 of the blockchain 2 receives the transaction and performs token payment processing. In addition, although the researcher pays a token to the patient who provides medical data in the present embodiment, the present invention is not limited thereto. For example, the researcher may pay a predetermined token only to a doctor who electronically signs medical data. Alternatively, the researcher may pay a predetermined token to both a patient who provides medical data and a doctor who electronically signs medical data. In addition, when a doctor electronically signs medical data despite the researcher's disclosure request, for example, a sponsor or a system operator may pay a predetermined token to the doctor.

In addition, although an example of a virtual currency (crypto asset), which is a type of token, has been described in the present embodiment, the present invention is not limited thereto. For example, other types of tokens such as electronic money, coupons or points can be similarly applied.

After performing the token payment processing, the node 21 acquires the hash value of the medical data based on the medical data ID. The node 21 transmits the acquired hash value of the medical data to the server 1. The server 1 receives the hash value of the medical data transmitted from the node 21. The server 1 acquires the hash value of the medical data from the medical data DB 174 based on the medical data ID. The server 1 compares the hash value of the medical data transmitted from the node 21 with the hash value of the medical data stored in the medical data DB 174. When the hash values match each other, the server 1 acquires medical data corresponding to the hash value of the medical data from the medical data DB 174. The server 1 transmits the acquired medical data to the researcher terminal 5. The researcher terminal 5 receives the medical data transmitted from the server 1 and displays the medical data on the screen.

In addition, a smart contract may be used without being limited to the token payment processing described above. The smart contract is a mechanism in which definitions of execution conditions and contract details are programmed in advance and incorporated into transactions and contracts are automatically fulfilled when a transaction that matches the execution conditions and the contract details occurs. The server 1 generates a smart contract code that describes the execution conditions, the wallet addresses of the patient and the researcher, and the like necessary for performing medical data transaction processing. The server 1 transmits a transaction including the generated smart contract code to any node 21 of the blockchain 2. The node 21 of the blockchain 2 records the received transaction including the smart contract.

In the content of the smart contract code, for example, a contract to pay a predetermined token from the researcher's wallet address to the patient's wallet address when the patient's approval for the researcher's disclosure request is obtained is defined in a code. The transaction includes the smart contract code and patient's and researcher's electronic signatures. The node 21 of the blockchain 2 pays a predetermined token determined in the contract from the researcher's wallet address to the patient's wallet address when the execution conditions approved by the patient for the researcher's disclosure request are met.

In addition, although an example in which a researcher pays a token to a patient has been described in the present embodiment, the present invention is not limited thereto, and a sponsor or a system operator may pay a predetermined token to the patient.

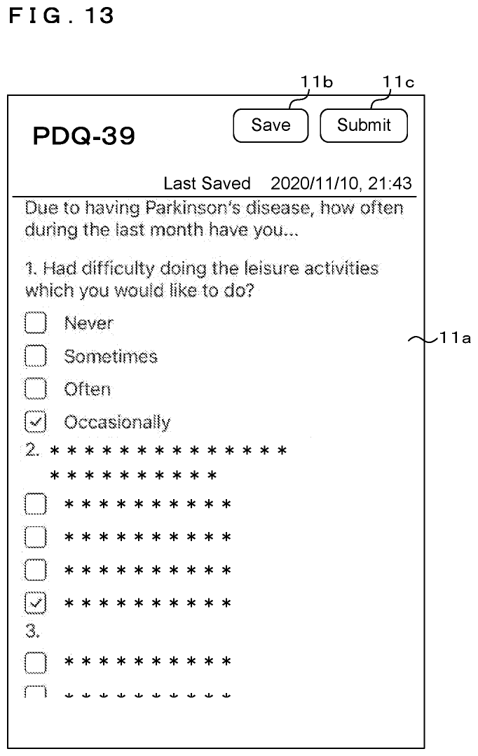

FIG. 13 is an explanatory diagram showing an example of an input screen for answers to questions. The screen includes an answer input field 11a, a save button 11b, and a submit button 11c. The answer input field 11a is an input field for receiving an input of a patient's answer to a medical question. The save button save 11b is a button for drafting and saving an answer to a question. The submit button 11c is a button for transmitting (submitting) an answer to a question to the server 1.

In addition, FIG. 13 illustrates an example in which the question type is PDQ-39, but the same applies to other question types. The patient terminal 4 acquires the PDQ-39 from the storage unit 12 of the server 1 or an external device. The patient terminal 4 displays the acquired PDQ-39 in the answer input field 11a. The patient terminal 4 receives the patient's answers to the questions when an input operation in the answer input field 11a is received. When a touch (click) operation on the save button 11b is received, the patient terminal 4 drafts and saves the answers input in the answer input field 11a. When a touch operation on the submit button 11c is received, the patient terminal 4 transmits the answers input in the answer input field 11a and the patient's attributes to the server 1 in association with the patient DID.

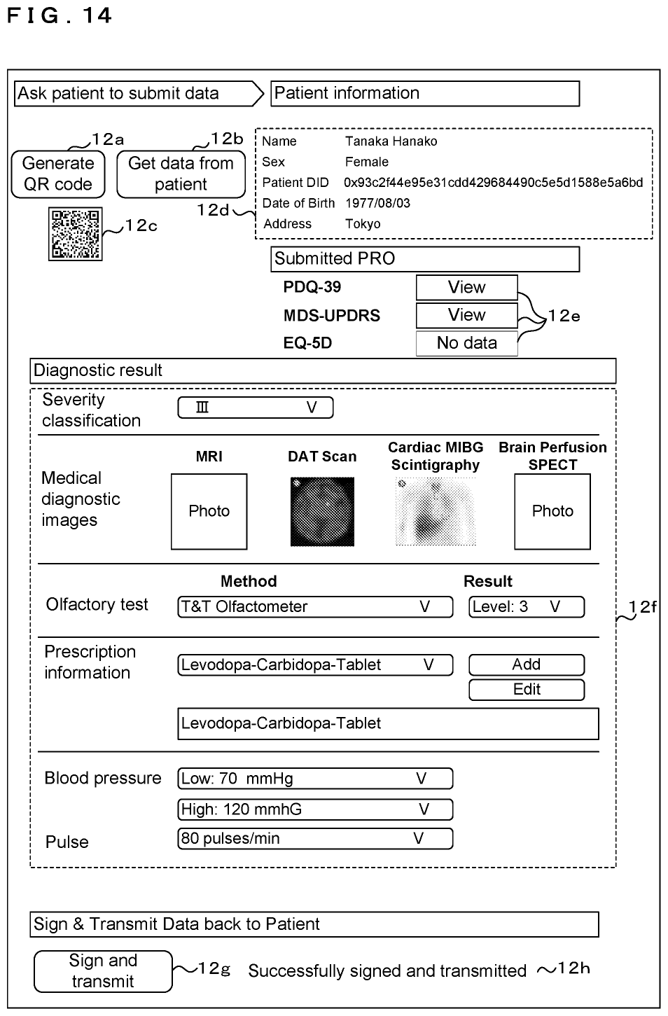

FIG. 14 is an explanatory diagram showing an example of a screen for creating medical data. The screen includes a QR code generation button 12a, a patient data acquisition button 12b, a QR code display field 12c, a patient attribute display field 12d, a view button 12e, a medical examination data reception field 12f, a signature and transmission button 12g, and a result display field 12h.

The QR code generation button 12a is a button for generating a QR code describing a doctor DID. The patient data acquisition button 12b is a button for acquiring patient's attributes and answers to questions from the patient. The QR code display field 12c is a display field for displaying a QR code. The patient attribute display field 12d is a display field for displaying the patient's attributes.

The view button 12e is a button for displaying answers to questions. In addition, the number of view buttons 12e may be provided based on the type of question. As shown in the diagram, the view button 12e is provided for each of “PDQ-39”, “MDS-UPDRS”, and “EQ-5D”. When it is determined that the answers to the questions have been acquired from the server 1, the doctor terminal 3 sets the title of the corresponding view button 12e to “view” to set the view button 12e to an active state in which the view button 12e can be operated. In addition, when it is determined that the answers to the questions have not been acquired from the server 1, the doctor terminal 3 sets the title of the corresponding view button 12e to “no data” and sets the view button 12e to an inactive state in which the view button 12e cannot be operated.

The medical examination data reception field 12f is a reception field for receiving an input of medical examination data obtained by a doctor diagnosing a patient. The signature and transmission button 12g is a button for electronically signing medical data including patient's attributes, answers to questions, and medical examination data, and transmitting the medical data. The result display field 12h is a display field for displaying the result of electronic signature and transmission processing for medical data.

When a click (touch) operation on the QR code generation button 12a is received, the doctor terminal 3 generates a QR code describing the doctor DID by using a library for generating a two-dimensional code. The doctor terminal 3 displays the generated QR code in the QR code display field 12c. The patient terminal 4 acquires the doctor DID by reading the QR code displayed on the doctor terminal 3.

The patient terminal 4 transmits the acquired doctor DID and patient DID to the server 1. The server 1 receives the doctor DID and the patient DID transmitted from the patient terminal 4, and acquires the patient's attributes and answers to questions from the patient DB 171 and question DB 173 based on the received patient DID. The server 1 transmits the acquired patient's attributes and answers to questions to the doctor terminal 3 corresponding to the received doctor DID.

In addition, the patient's attributes and the answers to questions can be acquired by the patient data acquisition button 12b. Specifically, the doctor terminal 3 generates a sub-screen for receiving an input of the patient DID when a click operation on the patient data acquisition button 12b is received. The doctor terminal 3 receives the input of the patient DID on the generated sub-screen, and transmits the received patient DID to the server 1. Thereafter, the patient's attributes and the answers to the questions are acquired from the server 1 in the same manner as in the processing described above.

The doctor terminal 3 receives the patient's attributes and the answers to the questions transmitted from the server 1. The doctor terminal 3 displays the received patient's attributes in the patient attribute display field 12d. When a click operation on the view button 12e is received, the doctor terminal 3 displays an answer to the corresponding question on the sub-screen. For example, when a click operation on the view button 12e corresponding to “PDQ-39” is received, the doctor terminal 3 generates a sub-screen for displaying the answer to the question. The doctor terminal 3 acquires answers to “PDQ-39” from the received answers to the questions, and displays the acquired answers to “PDQ-39” on the sub-screen.

The doctor terminal 3 receives an input operation in the medical examination data reception field 12f. The medical examination data includes diagnostic data, treatment data, and test data. The diagnostic data is data related to diagnosis, such as medical condition information and disease information (for example, a disease name determined by a doctor). The treatment data is data related to treatment, such as a treatment policy, a treatment method, surgery information, and drug information for the patient's disease. The test data is data related to various tests (for example, blood test) that the patient has undergone in order to check the presence or absence of a disease or the degree of the disease.

As shown in the diagram, the doctor terminal 3 receives, through the medical examination data reception field 12f, an input of diagnostic data including severity classification, treatment data including prescription information (ingredient names of drug, dosage form, dosage, and the like), and test data including medical diagnostic images, an olfactory test, blood pressure, and a pulse. The medical diagnostic images are images obtained by tests using, for example, MRI (Magnetic Resonance Image), DAT (Dopamine transporter) Scan, Cardiac MIBG (123I-metaiodobenzylguanidine) Scintigraphy, or Brain Perfusion SPECT (Single Photon Emission Computed Tomography). In addition, FIG. 14 illustrates an example of test items for Parkinson's disease, but the present invention is not limited thereto, and is similarly applied to test items for other types of diseases.

When a click operation on the signature and transmission button 12g is received, the doctor terminal 3 electronically signs the medical data including the patient's attributes, the answers to questions, and the medical examination data, and transmits the medical data with an electronic signature to the server 1 in association with the patient DID and the doctor DID. The doctor terminal 3 displays the results of the electronic signature and transmission processing on the medical data in the result display field 12h. As shown in the diagram, “successfully signed and transmitted” is displayed in the result display field 12h.

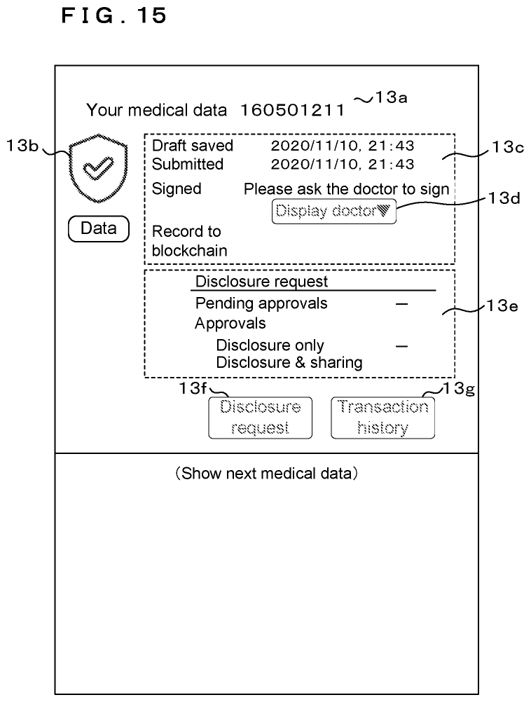

FIG. 15 is an explanatory diagram showing an example of a medical data display screen on the patient terminal 4 side. In addition, FIG. 15 is an example of medical data for which an electronic signature has not yet been acquired. The screen includes a medical data ID display field 13a, a status icon 13b, a date and time display field 13c, a doctor display button 13d, a disclosure request display field 13e, a disclosure request button 13f, and a transaction history display button 13g.

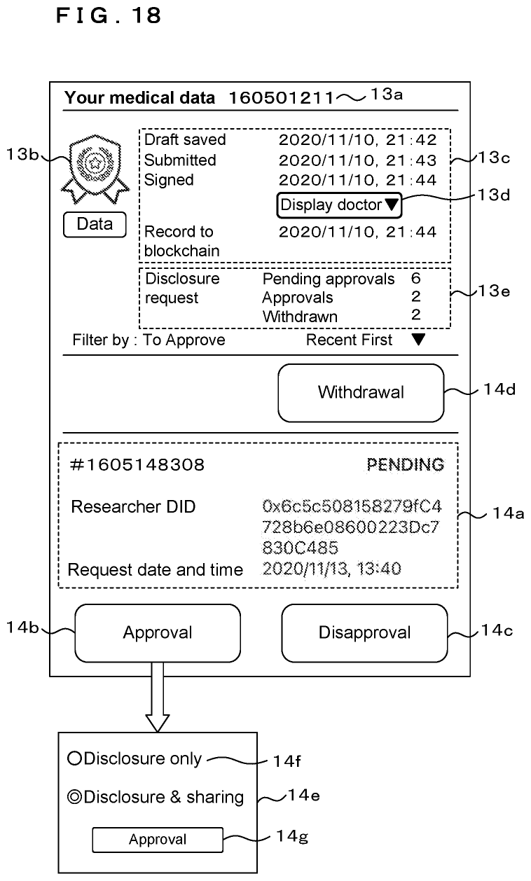

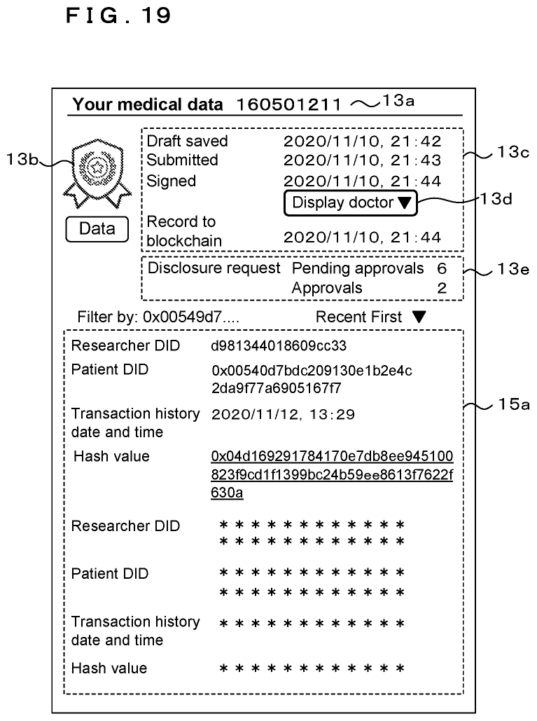

The medical data ID display field 13a is a display field for displaying the medical data ID. The status icon 13b is an icon indicating the status of medical data. The date and time display field 13c is a display field for displaying the update date and time of each status of medical data. The doctor display button 13d is a display field for displaying the attributes of a doctor who electronically signed the medical data. The disclosure request display field 13e is a display field for displaying information regarding the disclosure request. The disclosure request button 13f is a button for transitioning to a disclosure request response screen (FIG. 18), which will be described later. The transaction history display button 13g is a button for transitioning to a transaction history screen (FIG. 19), which will be described later.

The patient terminal 4 acquires information regarding the medical data from the management DB 177 of the server 1 based on the patient DID. The information regarding the medical data includes the medical data ID, the status of the medical data (draft saved, submitted, signed, and recorded in BC), and the update date and time of each status. The patient terminal 4 displays the information regarding the medical data on the screen for each medical data ID.

Specifically, the patient terminal 4 displays the medical data ID in the medical data ID display field 13a. The patient terminal 4 displays an icon corresponding to the status of the medical data in the status icon 13b. As shown in the diagram, when it is determined that the status of the medical data is “submitted”, the patient terminal 4 displays an icon indicating the “submitted” status in the status icon 13b and sets the doctor display button 13d to an inactive state in which the doctor display button 13d cannot be operated.

The patient terminal 4 displays the update date and time of each status in the date and time display field 13c. As shown in the diagram, the date and time of “draft saved”, the date and time of “submitted”, the date and time of “signed”, and the date and time of “recorded in BC” are displayed in the date and time display field 13c. In addition, since the electronic signature from the doctor has not yet been acquired, presentation information “please ask the doctor to sign” is displayed in the date and time display field 13c instead of the date and time “signed”. In addition, since the medical data is not recorded in the blockchain 2, “-” is displayed in the date and time display field 13c instead of the date and time of “recorded in BC”.

The patient terminal 4 sets the active state of the disclosure request button 13f and the transaction history display button 13g according to the status of the medical data. Specifically, when it is determined that the status of the medical data is “draft saved”, “submitted” or “signed”, the patient terminal 4 sets the disclosure request button 13f and the transaction history display button 13g to an inactive state in which the disclosure request button 13f and the transaction history display button 13g cannot be operated. When it is determined that the status of the medical data is “recorded in BC”, the patient terminal 4 sets the disclosure request button 13f and the transaction history display button 13g to an active state in which the disclosure request button 13f and the transaction history display button 13g can be operated.

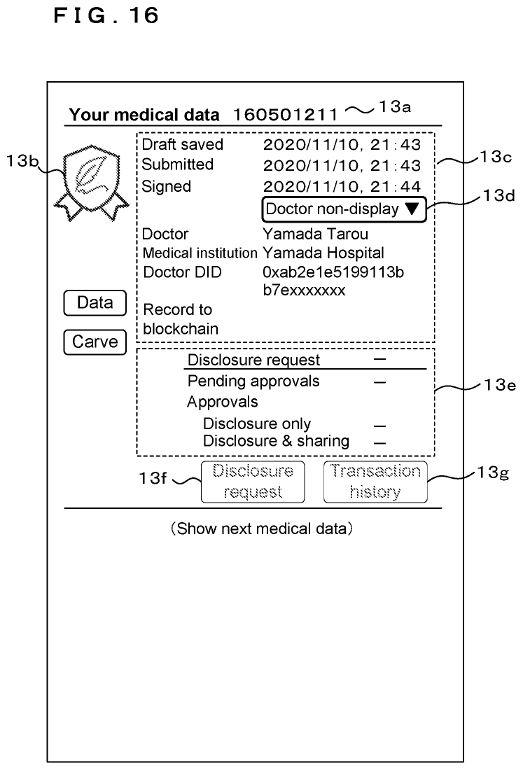

FIG. 16 is an explanatory diagram showing an example of a medical data display screen on the patient terminal 4 side. In addition, FIG. 16 is an example of medical data for which an electronic signature has been acquired. In addition, the same contents as in FIG. 15 are denoted by the same reference numerals, and the description thereof will be omitted.

The patient terminal 4 acquires information regarding the medical data from the management DB 177 of the server 1 based on the patient DID. The information regarding the medical data includes the medical data ID, the status of the medical data, the update date and time of each status, and the doctor DID and attributes of the doctor who electronically signed the medical data.

When it is determined that the status of the medical data is “signed”, the patient terminal 4 displays an icon indicating the “signed” status in the status icon 13b, and displays the date and time when the doctor has signed in the date and time display field 13c. The patient terminal 4 sets the doctor display button 13d to an active state in which the doctor display button 13d can be operated. When a touch operation on the doctor display button 13d is received, the patient terminal 4 transmits the medical data ID to the server 1. Based on the medical data ID transmitted from the patient terminal 4, the server 1 acquires the doctor DID of the doctor who signed the medical data from the medical data DB 174.

The server 1 acquires the attributes of the doctor from the doctor DB 172 based on the acquired doctor DID. The doctor's attributes include the name of the doctor and the name of the medical institution to which the doctor belongs. The server 1 transmits the acquired doctor DID and doctor's attributes to the patient terminal 4. The patient terminal 4 receives the doctor DID and the doctor's attributes transmitted from the server 1, displays the received doctor DID and the doctor's attributes in the date and time display field 13c (area between the date and time of “signed” and the date and time of “recorded in BC”), and changes the title of the doctor display button 13d to “doctor non-display”.

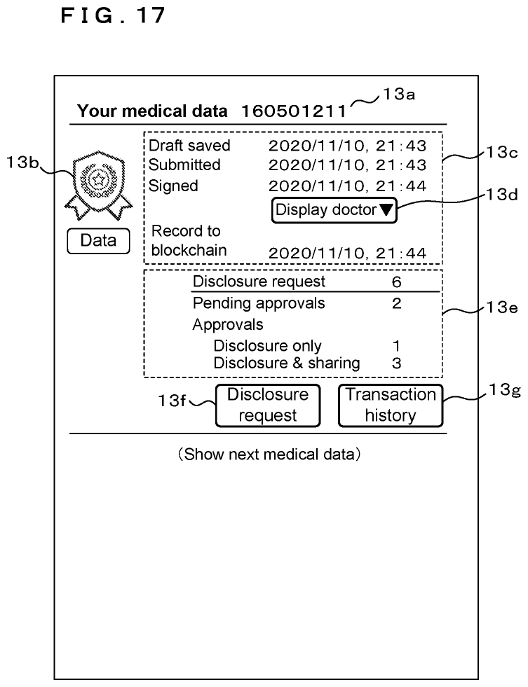

FIG. 17 is an explanatory diagram showing an example of a medical data display screen on the patient terminal 4 side. In addition, FIG. 17 is an example of medical data recorded in the blockchain 2. In addition, the same contents as in FIGS. 15 and 16 are denoted by the same reference numerals, and the description thereof will be omitted.

The patient terminal 4 acquires information regarding medical data and information regarding a disclosure request from the server 1 based on the patient DID. The information regarding the medical data includes the medical data ID, the status of the medical data, the update date and time of each status, and the doctor DID and attributes of the doctor who electronically signed the medical data. The information regarding the disclosure request includes the number of requests for disclosure of medical data, the number of pending approvals, the number of approvals for “disclosure only”, and the number of approvals for “disclosure & sharing”.

Specifically, the patient terminal 4 transmits the patient DID to the server 1. The server 1 receives the patient DID transmitted from the patient terminal 4. The server 1 acquires information regarding the medical data from the management DB 177 based on the received patient DID. The server 1 acquires information regarding the medical data disclosure request from the management DB 177 based on the patient DID and the medical data ID.

The server 1 transmits the acquired information regarding the medical data and the acquired information regarding the disclosure request to the patient terminal 4. The patient terminal 4 receives the information regarding the medical data and the information regarding the disclosure request transmitted from the server 1 and displays these on the screen. In addition, since the processing for displaying the information regarding the medical data is the same as that in FIG. 16, the description thereof will be omitted. The patient terminal 4 displays the received information regarding the disclosure request in the disclosure request display field 13e. As shown in the diagram, the number of requests for disclosure of medical data, the number of pending approvals, the number of approvals for “disclosure only”, and the number of approvals for “disclosure & sharing” are displayed in the disclosure request display field 13e.

When it is determined that the status of the medical data is “recorded in BC”, the patient terminal 4 sets an icon indicating the “recorded in BC” status in the status icon 13b and displays the date and time of “recorded in BC” in the date and time display field 13c, and sets the disclosure request button 13f and the transaction history display button 13g to an active state in which the disclosure request button 13f and the transaction history display button 13g can be operated. When a touch operation on the disclosure request button 13f is received, the patient terminal 4 transitions to a disclosure request response screen (FIG. 18), which will be described later. When a touch operation on the transaction history display button 13g is received, the patient terminal 4 transitions to a medical data transaction history screen (FIG. 19), which will be described later.

FIG. 18 is an explanatory diagram showing an example of a disclosure request response screen on the patient terminal 4 side. In addition, the same contents as in FIGS. 15 to 17 are denoted by the same reference numerals, and the description thereof will be omitted. The screen includes a disclosure request display field 14a, an approval button 14b, a disapproval button 14c, a withdrawal button 14d, and an approval type setting dialog 14e.

The disclosure request display field 14a is a display field for displaying information regarding a medical data disclosure request. The approval button 14b is a button for performing approval processing in response to a medical data disclosure request. The disapproval button 14c is a button for performing disapproval processing in response to a medical data disclosure request. The withdrawal button 14d is a button for withdrawing the approval for approved medical data. The approval type setting dialog 14e is a dialog (child screen) for receiving the setting of approval type for medical data.

The patient terminal 4 acquires information regarding the medical data disclosure request from the server 1 based on the medical data ID. The patient terminal 4 displays the acquired information regarding the disclosure request in the disclosure request display field 14a. As shown in the diagram, a researcher DID, application date and time, and a request status (for example, waiting for approval) are displayed in the disclosure request display field 14a. In addition, the information regarding the disclosure request is not limited to the above items, and may include, for example, the name and e-mail address of the researcher.

When a touch operation on the approval button 14b is received, the patient terminal 4 generates the approval type setting dialog 14e and displays the approval type setting dialog 14e on the screen. The approval type setting dialog 14e includes an approval type setting radio button 14f and an intra-dialog approval button 14g. The approval type setting radio button 14f is a radio button for receiving the approval type (“disclosure only” or “disclosure & sharing”) for medical data. The intra-dialog approval button 14g is a button for performing approval processing of medical data according to the approval type.

When a touch operation on the approval type setting radio button 14f is received, the patient terminal 4 receives the setting of the approval type. When a touch operation on the intra-dialog approval button 14g is received, the patient terminal 4 transmits the approval type set by the approval type setting radio button 14f to the server 1 in association with the medical data ID.

When a touch operation on the disapproval button 14c is received, the patient terminal 4 transmits disapproval information indicating that the use of the medical data has been disapproved to the server 1 in association with the medical data ID. When a touch operation on the withdrawal button 14d is received, the patient terminal 4 transmits approval withdrawal information indicating that the approval for the approved medical data has been withdrawn to the server 1 in association with the medical data ID.

FIG. 19 is an explanatory diagram showing an example of a medical data transaction history screen on the patient terminal 4 side. In addition, the same contents as in FIGS. 15 to 17 are denoted by the same reference numerals, and the description thereof will be omitted. The screen includes a transaction history display field 15a. The transaction history display field 15a is a display field for displaying the transaction history of medical data.

The patient terminal 4 transmits the patient DID and the medical data ID to the server 1. The server 1 receives the patient DID and the medical data ID transmitted from the patient terminal 4. The server 1 acquires the medical data transaction history from the transaction history DB 176 based on the received patient DID and medical data ID. The server 1 transmits the acquired transaction history to the patient terminal 4. The patient terminal 4 receives the transaction history transmitted from the server 1 and displays the transaction history in the transaction history display field 15a. As shown in the diagram, a transaction history including the researcher DID, the patient DID, the transaction history date and time, and the hash value of the medical data is displayed in the transaction history display field 15a.

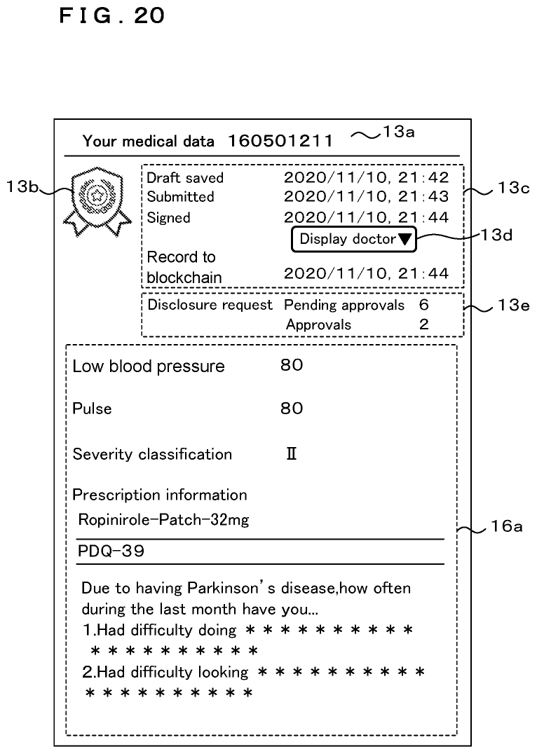

FIG. 20 is an explanatory diagram showing an example of a detailed medical data screen on the patient terminal 4 side. In addition, the same contents as in FIGS. 15 to 17 are denoted by the same reference numerals, and the description thereof will be omitted. The screen includes a medical data display field 16a. The medical data display field 16a is a display field for displaying detailed information of medical data.

The patient terminal 4 transmits the patient DID and the medical data ID to the server 1. The server 1 receives the patient DID and the medical data ID transmitted from the patient terminal 4. Based on the received patient DID and medical data ID, the server 1 acquires, from the medical data DB 174, medical data including the patient's attributes, the patient's answers to medical questions (for example, answers to PDQ-39), and the doctor's medical examination data for the patient (for example, blood pressure, prescription information, or pulse). The server 1 transmits the acquired medical data to the patient terminal 4. The patient terminal 4 receives the medical data transmitted from the server 1 and displays the medical data in the medical data display field 16a.

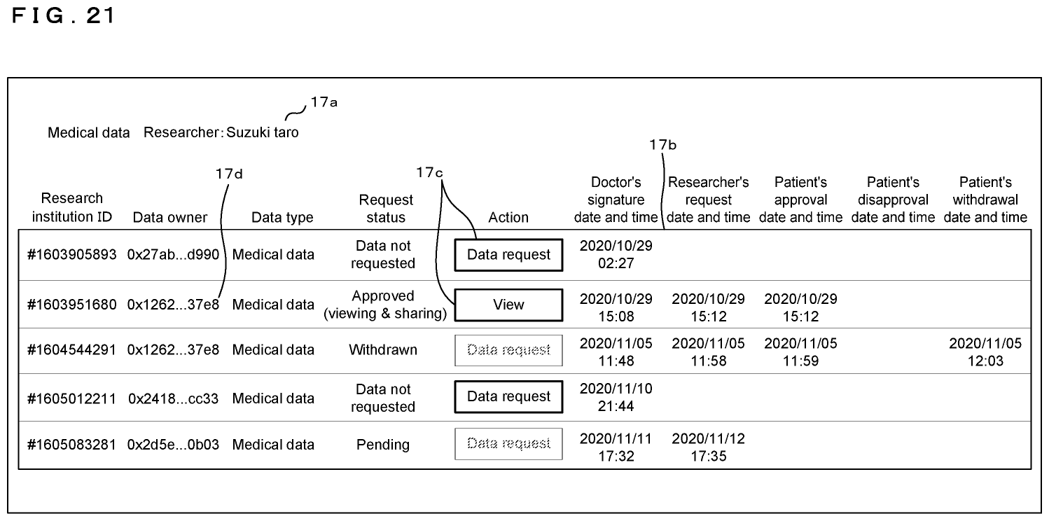

FIG. 21 is an explanatory diagram showing an example of a medical data management screen on the researcher terminal 5 side. The screen includes a researcher display field 17a, a management information display field 17b, an action button 17c, and a patient DID link 17d. The researcher display field 17a is a display field for displaying the name of the researcher. The management information display field 17b is a display field for displaying management information of medical data.

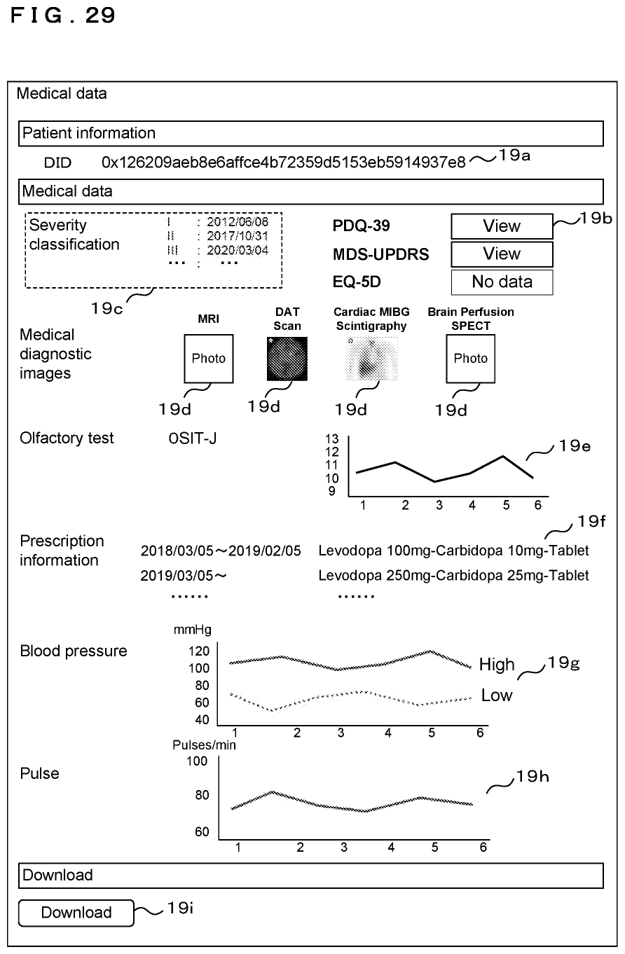

The action button 17c is a button for receiving an action on medical data. Actions include “data request” and “view”. The “data request” action is an action of transmitting a medical data disclosure request to a patient who provided the medical data. The “view” action is an action of viewing (displaying) medical data. In addition, although an example in which actions include “data request” and “view” has been described in the present embodiment, the present invention is not limited thereto. For example, actions may include “re-approval” or “re-request”. The “re-approval” action is an action of transmitting a request for approval again for medical data whose disclosure was once rejected in response to a disclosure request from the researcher. The “re-request” action is an action of re-transmitting a request for disclosure of medical data to the patient who provided the medical data after a disclosure period (for example, one year) has passed if the disclosure period is set for the medical data. The patient DID link 17d is a button for transitioning to a medical data display screen (FIG. 29), which will be described later.

The researcher terminal 5 transmits the researcher DID to the server 1. The server 1 receives the researcher DID transmitted from the researcher terminal 5, and acquires the researcher's name and research institution ID from the researcher DB 175 based on the received researcher DID. Based on the researcher DID, the server 1 acquires from the management DB 177 the patient DID, information regarding the disclosure request, and the date and time of the doctor's signature for the medical data. The server 1 transmits to the researcher terminal 5 the name of the researcher, the research institution ID, the patient DID, the information regarding the disclosure request, and the date and time of the doctor's signature for the medical data that have been acquired.

The researcher terminal 5 receives the name of the researcher, the research institution ID, the patient DID, the information regarding the disclosure request, and the signature date and time of the medical data transmitted from the server 1. The researcher terminal 5 displays the received name of the researcher in the researcher display field 17a, and displays the research institution ID, the patient DID, the information regarding the disclosure request, and the signature date and time of the medical data in the management information display field 17b. The information regarding the disclosure request includes data type, disclosure request status, disclosure request date and time, approval date and time, disapproval date and time, and approval withdrawal date and time. The disclosure request status includes data not requested, approved (viewing & sharing), approved (viewing only), withdrawn and pending.

As shown in the diagram, the research institution ID, patient DID (data owner), data type, disclosure request (request) status, action button 17c generated according to the disclosure request status, doctor's signature date and time, researcher's disclosure request (request) date and time, patient's approval date and time, patient's disapproval date and time, and patient's approval withdrawal date and time are displayed in the management information display field 17b.

The researcher terminal 5 sets the title and active state of the action button 17c according to the disclosure request status. Specifically, when it is determined that the disclosure request status is “data not requested”, the researcher terminal 5 sets the title of the action button 17c to “data request”, and sets the action button 17c to an active state in which the action button 17c can be operated. When it is determined that the disclosure request status is “withdrawn” or “pending”, the researcher terminal 5 sets the title of the action button 17c to “data request”, and sets the action button 17c to an inactive state in which the action button 17c cannot be operated. When it is determined that the disclosure request status is “approved (viewing & sharing)” or “approved (viewing only)”, the researcher terminal 5 sets the title of the action button 17c to “view”, and sets the action button 17c to an active state in which the action button 17c can be operated.

The researcher terminal 5 transmits the researcher DID and the medical data ID to the server 1 when a touch operation on the action button 17c whose title is “data request” is received. The server 1 receives the researcher DID and the medical data ID transmitted from the researcher terminal 5, and creates a request for disclosure of the medical data based on the received researcher DID and medical data ID. The server 1 transmits the created disclosure request to the patient terminal 4.

The researcher terminal 5 acquires the medical data from the medical data DB 174 of the server 1 based on the medical data ID when a touch operation on the action button 17c whose title is “view” is received. The researcher terminal 5 displays the acquired medical data on the screen. Once the patient approves the disclosure of the medical data to the researcher, the medical data can be viewed continuously. Alternatively, a disclosure period may be set for the medical data. For example, a researcher can repeatedly view the medical data within a predetermined period (for example, one year).

When a click operation on the patient DID link 17d is received, the researcher terminal 5 determines the disclosure request status. When it is determined that the status of the disclosure request is “approved (viewing & sharing)” or “approved (viewing only)”, the researcher terminal 5 transitions to a display screen (FIG. 29) of medical data owned by the patient corresponding to the patient DID.

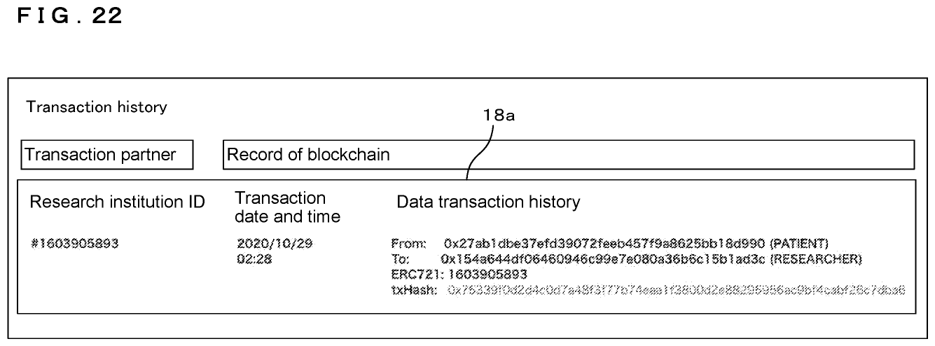

FIG. 22 is an explanatory diagram showing an example of a transaction history screen on the researcher terminal 5 side. The screen includes a transaction history display field 18a. The transaction history display field 18a is a display field for displaying the transaction history. The researcher terminal 5 transmits the researcher DID to the server 1. The server 1 receives the researcher DID transmitted from the researcher terminal 5 and acquires the researcher's medical data transaction history from the transaction history DB 176 based on the received researcher DID. The server 1 transmits the acquired transaction history to the researcher terminal 5.

The researcher terminal 5 receives the transaction history transmitted from the server 1 and displays the transaction history in the transaction history display field 18a. As shown in the diagram, the medical data transaction history including the research institution ID of the research institution to which the researcher belongs, the medical data ID, the transaction date and time, the patient DID, the researcher DID, and the hash value of the medical data is displayed in the transaction history display field 18a. In addition, although the display of a single transaction history is exemplified, the present invention is not limited thereto, and a plurality of transaction histories may be listed.

In addition, the medical data transaction history described above may be transmitted to the doctor terminal 3 or the patient terminal 4, and the transaction history screen may be displayed on the doctor terminal 3 side or the patient terminal 4 side.

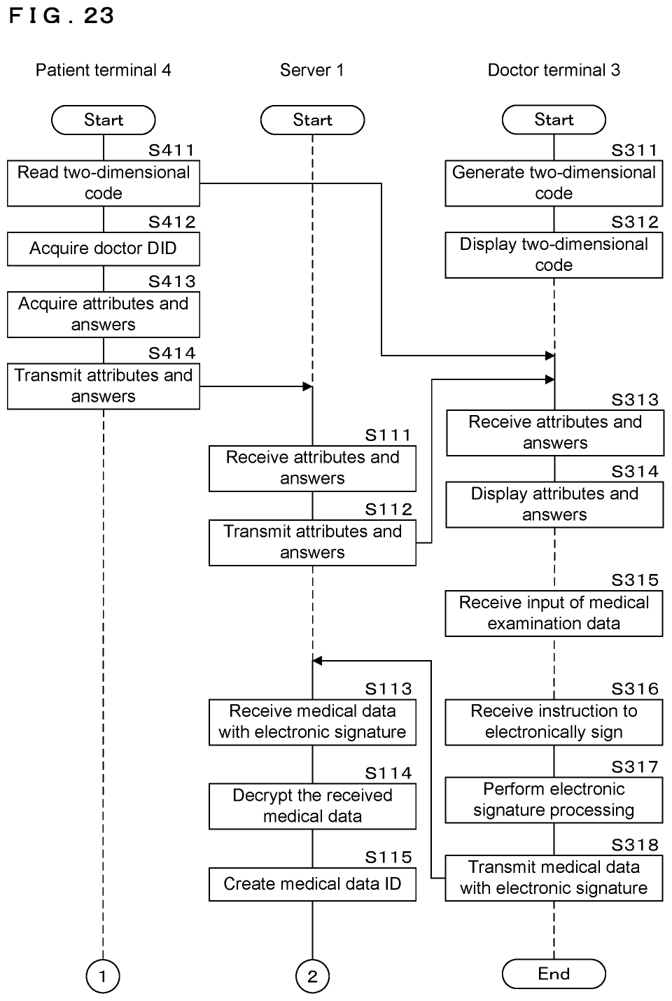

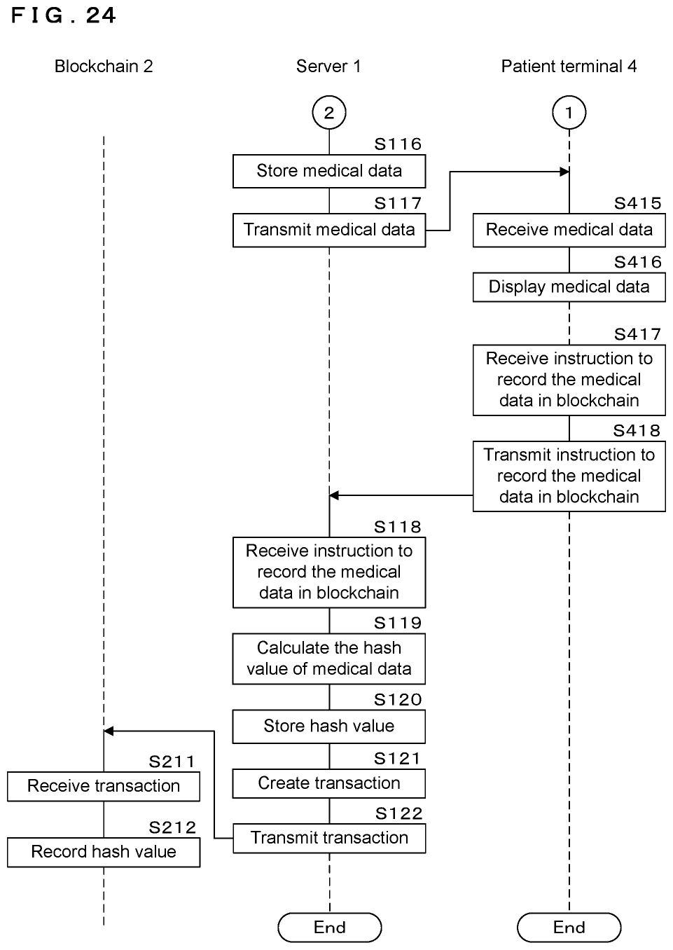

FIGS. 23 and 24 are flowcharts showing a processing procedure when creating medical data. The control unit 31 of the doctor terminal 3 generates a two-dimensional code (for example, a QR code) describing the doctor DID by using a library for generating a two-dimensional code (step S311). The control unit 31 displays the generated two-dimensional code on the display unit 35 (step S312). The control unit 41 of the patient terminal 4 reads the two-dimensional code displayed on the doctor terminal 3 by using the imaging unit 46 (step S411).

The control unit 41 acquires the doctor DID from the read two-dimensional code by using a two-dimensional code analysis library (step S412). The control unit 41 acquires the patient's attributes and the patient's answers to medical questions from the server 1 through the communication unit 43 (step S413). Specifically, based on the patient DID, the control unit 41 acquires the patient's attributes from the patient DB 171 of the mass storage unit 17 of the server 1, and acquires the answers to the questions from the question DB 173 of the mass storage unit 17 of the server 1. In addition, the control unit 41 may receive an input of an answer to a question by the patient through the input unit 44.

The control unit 41 transmits the acquired patient's attributes and answers to questions to the server 1 through the communication unit 43 in association with the patient DID and the doctor DID (step S414). The control unit 11 of the server 1 receives the patient DID, the doctor DID, the patient's attributes, and the answers transmitted from the patient terminal 4 through the communication unit 13 (step S111), and transmits the received patient DID, doctor DID, patient's attribute, and answers to the doctor terminal 3 (step S112). The control unit 31 of the doctor terminal 3 receives the patient DID, the doctor DID, the patient's attributes, and the answers transmitted from the server 1 through the communication unit 33 (step S313). The control unit 31 displays the received patient attributes and answers on the display unit 35 (step S314).

The control unit 31 receives an input of medical examination data for the patient by the doctor through the input unit 34 (step S315). The control unit 31 receives an instruction to electronically sign medical data including the patient's attributes, answers to questions, and medical examination data through the input unit 34 (step S316), and the electronic signature creation unit 31b of the control unit 31 performs electronic signature processing on the medical data by using the doctor's private key (step S317). The control unit 31 transmits the medical data with an electronic signature to the server 1 through the communication unit 33 in association with the patient DID and the doctor DID (step S318).

The control unit 11 of the server 1 receives the patient DID, the doctor DID, and the medical data with an electronic signature, which are transmitted from the doctor terminal 3, through the communication unit 13 (step S113). The control unit 11 decrypts the received medical data with an electronic signature by using the public key (step S114). The control unit 11 creates a medical data ID for identifying the medical data by using, for example, ERC721, which is one smart contract standard of Ethereum (step S115). The control unit 11 stores the patient DID, the medical data, the doctor DID, the date and time of the electronic signature, and the status of the “signed” medical data, as one record, in the medical data DB 174 in association with the created medical data ID (step S116).

The control unit 11 transmits the medical data to the patient terminal 4 through the communication unit 13 (step S117). The control unit 41 of the patient terminal 4 receives the medical data transmitted from the server 1 through the communication unit 43 (step S415), and displays the received medical data on the display unit 45 (step S416). When an instruction from the patient to record the medical data in the blockchain 2 is received through the input unit 44 (step S417), the control unit 41 transmits the instruction to record the medical data in the blockchain 2 to the server 1 through the communication unit 43 (step S418).

The control unit 11 of the server 1 receives the instruction to record the medical data in the blockchain 2, which is transmitted from the patient terminal 4, through the communication unit 13 (step S118). The hash value calculation unit 11a of the control unit 11 calculates the hash value of the medical data by using a cryptographic hash function (step S119). The control unit 11 stores the calculated hash value of the medical data in the medical data DB 174 in association with the medical data ID (step S120). The control unit 11 creates a transaction by associating the calculated hash value of the medical data with the patient DID and the doctor DID (step S121). The created transaction includes the patient DID, the doctor DID, and the hash value of the medical data.

The control unit 11 transmits the created transaction to one of the nodes 21 of the blockchain 2, which manages the transaction data in a decentralized manner, through the communication unit 13 (step S122). The control unit 211 of the node 21 of the blockchain 2 receives the transaction transmitted from the server 1 through the communication unit 213 (step S211). Based on the received transaction, the control unit 211 of the node 21 records the hash value of the medical data in association with the patient DID and the doctor DID (step S212).

Specifically, the control unit 211 of the node 21 that has received the transaction generates, through the block generation unit 216, a block including at least previous block management information created based on information of the blockchain (ledger) managed by itself and the patient DID, the doctor DID, and the hash value of the medical data included in the received transaction. Then, the control unit 211 of each node 21 of the blockchain 2 performs predetermined consensus processing by using the block generation unit 216. A blockchain is a time-series arrangement of data groups having a predetermined data structure called blocks, and serves as a ledger in which transaction details are recorded. Each block includes information of blocks located before the block by one or more blocks (hereinafter, referred to as previous blocks) and information (nonce, signature, and the like) for detecting whether or not there has been tampering, in addition to data to be recorded in the block, such as transaction details. The nonce is a value in a predetermined data area that is set so that a value obtained when the data in the data area is processed by a one-way function satisfies a predetermined rule.

A new block is added to the blockchain 2 through processing according to a predetermined consensus algorithm in which two or more nodes participate. For example, in PoW, which is one of the consensus algorithms, a rule such as “the Hash value of the block should be equal to or less than a threshold value” is determined in advance for each block (data group) including the information of the previous block and the nonce. When adding a block, processing in which a plurality of nodes concurrently searches for a nonce that satisfies such a rule is performed by the block verification unit 217. In addition, other than PoW, there are consensus algorithms such as BFT (Byzantine fault tolerance). After performing predetermined consensus processing, the control unit 211 of each node 21 of the blockchain 2 stores (adds) the block generated by the block generation unit 216 in the storage unit 212 through the block sharing unit 218.

In addition, although the processing for recording in the blockchain 2 is performed on the server 1 side in the present embodiment, the present invention is not limited thereto, and the processing for recording in the blockchain 2 may be performed on the patient terminal 4 side.

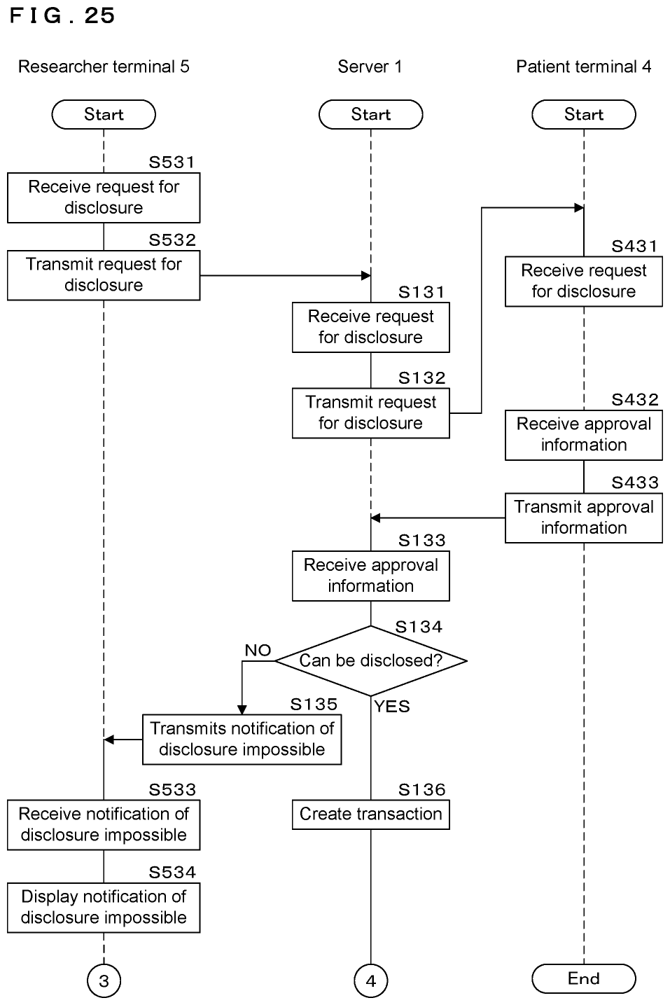

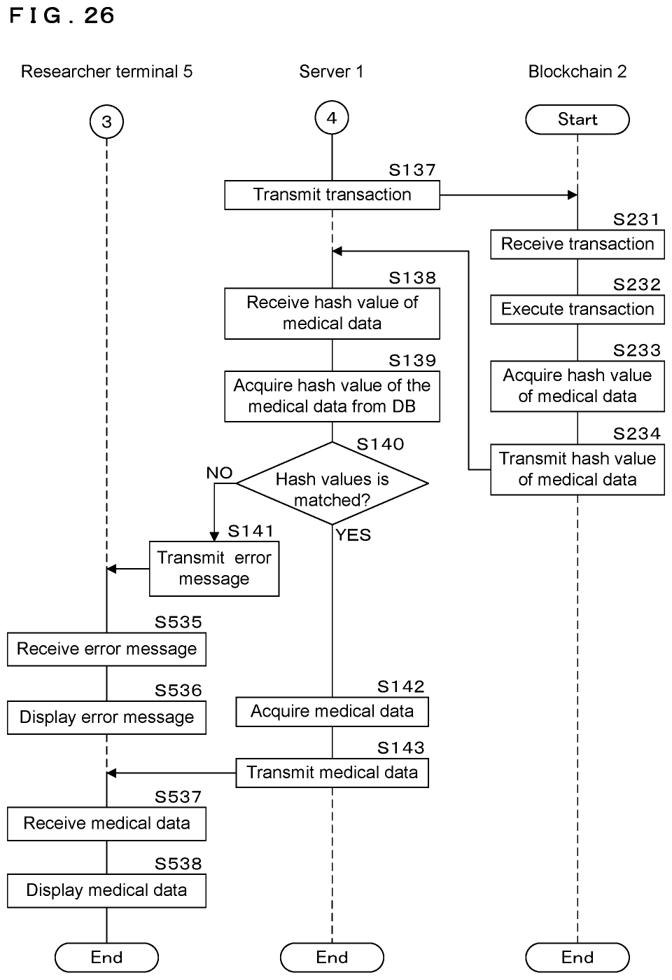

FIGS. 25 and 26 are flowcharts showing a processing procedure when outputting medical data approved by the patient to the researcher terminal 5. The control unit 51 of the researcher terminal 5 receives a request for disclosure of medical data recorded in the blockchain 2 through the input unit 54 (step S531). The control unit 51 transmits the received disclosure request to the server 1 through the communication unit 53 in association with the researcher DID and the medical data ID (step S532). The control unit 11 of the server 1 receives the researcher DID, the medical data ID, and the disclosure request transmitted from the researcher terminal 5 through the communication unit 13 (step S131). The control unit 11 transmits the received researcher ID, medical data ID, and disclosure request to the patient terminal 4 of the patient who provided the medical data (step S132).

The control unit 41 of the patient terminal 4 receives the researcher ID, medical data ID, and disclosure request transmitted from the server 1 through the communication unit 43 (step S431). In response to the received disclosure request, the control unit 41 receives approval information for the medical data (step S432), and transmits the received approval information to the server 1 through the communication unit 43 (step S433). The approval information includes information indicating whether or not the medical data can be disclosed and the approval type (“disclosure only” or “disclosure & sharing”) for the medical data.

The control unit 11 of the server 1 receives the approval information transmitted from the patient terminal 4 through the communication unit 13 (step S133). Based on the received approval information, the control unit 11 determines whether or not the medical data, which is a target of the disclosure request, can be disclosed (step S134). When it is determined that the medical data cannot be disclosed (NO in step S134), the control unit 11 transmits a notification indicating that the medical data cannot be disclosed to the researcher terminal 5 through the communication unit 13 (step S135). The control unit 51 of the researcher terminal receives the notification of disclosure impossible transmitted from the server 1 through the communication unit 53 (step S533), and displays the received notification of disclosure impossible on the display unit 55 (step S534).

When it is determined that the medical data can be disclosed (YES in step S134), the control unit 11 creates a transaction for paying a predetermined token from the researcher to the patient (step S136). The transaction includes the researcher's wallet address, the patient's wallet address, the amount of tokens paid, and the date and time of transaction. The control unit 11 transmits the created transaction to one of the nodes 21 of the blockchain 2 through the communication unit 13 (step S137).

The node 21 of the blockchain 2 receives the transaction transmitted from the server 1 through the communication unit 213 (step S231), and executes the received transaction (step S232). The control unit 211 of the node 21 acquires the hash value of the medical data based on the medical data ID (step SS233). The control unit 211 of the node 21 transmits the acquired hash value of the medical data to the server 1 through the communication unit 213 (step S234).

The control unit 11 of the server 1 receives the hash value of the medical data transmitted from the blockchain 2 through the communication unit 13 (step S138). The control unit 11 acquires the hash value of the medical data from the medical data DB 174 of the mass storage unit 17 based on the medical data ID (step S139). The control unit 11 compares the acquired hash value of the medical data with the hash value of the medical data transmitted from the node 21 to determine whether or not the hash values match each other (step S140).

When it is determined that the hash values do not match each other (NO in step S140), the control unit 11 transmits an error message indicating that the medical data is invalid to the researcher terminal 5 through the communication unit 13 (step S141). The control unit 51 of the researcher terminal 5 receives the error message transmitted from the server 1 through the communication unit 53 (step S535), and displays the received error message on the display unit 55 (step S536).

When it is determined that the hash values match each other (YES in step S140), the control unit 11 acquires medical data corresponding to the hash value of the medical data from the medical data DB 174 of the mass storage unit 17 (step S142). The control unit 11 transmits the acquired medical data to the researcher terminal 5 through the communication unit 13 (step S143). The control unit 51 of the researcher terminal 5 receives the medical data transmitted from the server 1 through the communication unit 53 (step S537), displays the received medical data on the display unit 55 (step S538), and ends the process.

According to the present embodiment, it is possible to create medical data with an electronic signature by a doctor.

According to the present embodiment, researchers can actively promote the provision of medical data by paying a predetermined token to a patient who provides the medical data.

According to the present embodiment, researchers can conduct medical research using medical data recorded in the blockchain 2, thereby contributing to the research of new treatment methods, the extension of healthy life expectancy, the improvement of the quality of medical services, the suppression of medical expenses or nursing care expenses, and the like.

### Second Embodiment

A second embodiment relates to a form in which medical data is shared between researchers. In addition, the description of the same contents as in the first embodiment will be omitted.

Researchers use medical data based on the approval type of medical data set by the patient. When the approval type of medical data is “disclosure only”, the medical data can be disclosed to the researcher who made a disclosure request, but cannot be shared with other researchers. When the approval type of medical data is “disclosure & sharing”, the medical data can be disclosed to the researcher who made a disclosure request, and can be shared with other researchers.

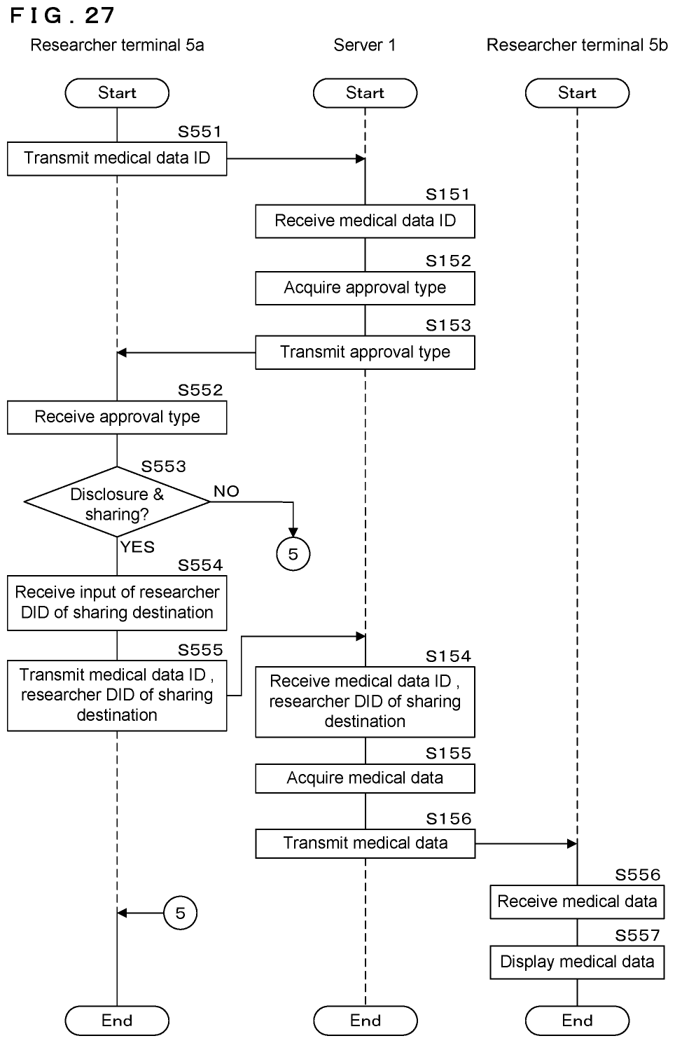

FIG. 27 is a flowchart showing a processing procedure when sharing medical data. In addition, in the flowchart of FIG. 27, for convenience of explanation, the researcher terminal 5 that is a sharing source is denoted by reference numeral 5a, and the researcher terminal 5 that is a sharing destination is denoted by reference numeral 5b.

The control unit 51 of the researcher terminal 5a transmits the medical data ID to the server 1 through the communication unit 53 (step S551). The control unit 11 of the server 1 receives the medical data ID transmitted from the researcher terminal 5a through the communication unit 13 (step S151). Based on the received medical data ID, the control unit 11 acquires the approval type of the medical data from the medical data DB 174 of the mass storage unit 17 (step S152). The control unit 11 transmits the acquired approval type to the researcher terminal 5a through the communication unit 13 (step S153).

The control unit 51 of the researcher terminal 5a receives the approval type transmitted from the server 1 through the communication unit 53 (step S552). The control unit 51 determines whether or not the received approval type is “disclosure & sharing” (step S553). When it is determined that the approval type is not “disclosure & sharing” (NO in step S553), the control unit 51 ends the process. When it is determined that the approval type is “disclosure & sharing” (YES in step S553), the control unit 51 receives an input of the researcher DID of the sharing destination through the input unit 54 (step S554).

The control unit 51 transmits the medical data ID and the received researcher DID of the sharing destination to the server 1 through the communication unit 53 (step S555). The control unit 11 of the server 1 receives, through the communication unit 13, the medical data ID and the researcher DID of the sharing destination transmitted from the researcher terminal 5a (step S154). Based on the received medical data ID, the control unit 11 acquires the medical data from the medical data DB 174 of the mass storage unit 17 (step S155). The control unit 11 transmits the acquired medical data to the researcher terminal 5b through the communication unit 13 (step S156). The control unit 51 of the researcher terminal 5b receives the medical data transmitted from the server 1 through the communication unit 53 (step S556), displays the received medical data on the display unit 55 (step S557), and ends the process.

In addition, the present invention is not limited to the sharing process described above. For example, when medical data whose approval type is “disclosure & sharing” is disclosed to a researcher, the medical data may be automatically shared with other researchers in the research institution to which the researcher belongs.

According to the present embodiment, it is possible to help utilize medical data by sharing the medical data with other researchers.

### Third Embodiment

A third embodiment relates to a form in which information regarding the results of research conducted by using medical data is output to the patient terminal 4. In addition, the description of the same contents as in the first and second embodiments will be omitted. Research results include, for example, academic papers, reports, and research plans based on medical data of a large number of patients, videos of diseases, symptoms, and treatment methods related to treatment, and the like.

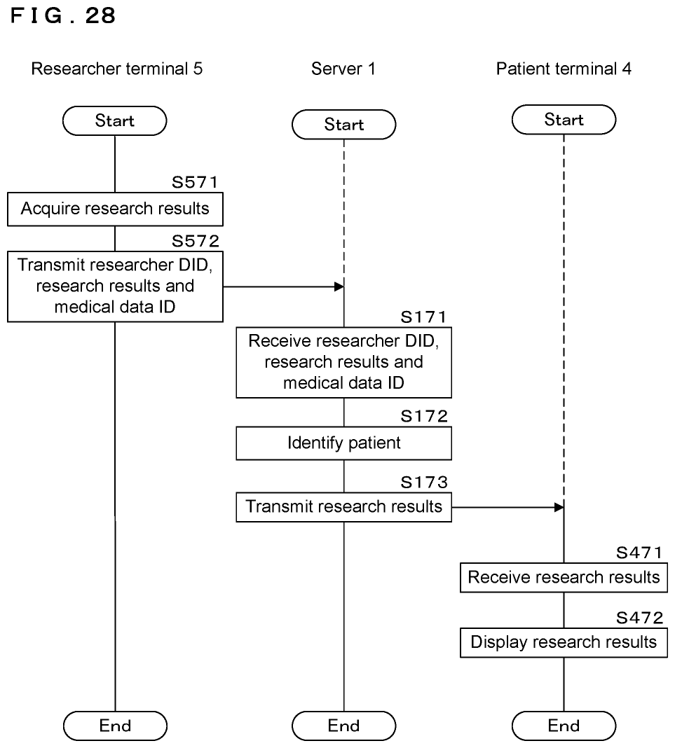

FIG. 28 is a flowchart showing a processing procedure when outputting information regarding research results to the patient terminal 4. The control unit 51 of the researcher terminal 5 acquires information regarding the results of research conducted by using medical data (step S571). Specifically, the control unit 51 may receive information regarding research results through the input unit 54, or may acquire the information regarding research results from an external device through the communication unit 53. For example, if the research results are a paper, the information regarding the research results includes the title of the paper, author/editor, bibliography number, publication date and time, content, and the like.

The control unit 51 transmits the researcher DID and the acquired information regarding the research results to the server 1 through the communication unit 53 in association with the medical data ID associated with the research results (step S572). The control unit 11 of the server 1 receives, through the communication unit 13, the researcher DID, the information regarding the research results, and the medical data ID transmitted from the researcher terminal 5 (step S171). Based on the received medical data ID, the control unit 11 identifies the patient who provided the medical data (step S172). Specifically, based on the researcher DID and the medical data ID, the control unit 11 acquires the patient DID from the transaction history DB 176 of the mass storage unit 17 to identify the patient.

The control unit 11 transmits the information regarding the research results to the patient terminal 4 of the identified patient through the communication unit 13 (step S173). The control unit 41 of the patient terminal 4 receives the information regarding the research results transmitted from the server 1 through the communication unit 43 (step S471), displays the received information regarding the research results on the display unit 45 (step S472), and ends the process.

According to the present embodiment, it is possible to actively promote the provision of medical data from the patient by sharing (transmitting) the results of research using the medical data with the patient who provided the medical data.

### Fourth Embodiment

A fourth embodiment relates to a form in which multiple times of medical data is output in an integrated manner. In addition, the description of the same contents as in the first to third embodiments will be omitted. In the present embodiment, processing for outputting to the researcher terminal 5 medical data including patient's attributes, multiple times of patient's answers to questions, and diagnosis data obtained by integrating diagnosis data at the time of the first diagnosis by a doctor and diagnosis data at the time of the second and subsequent diagnoses will be described.

FIG. 29 is an explanatory diagram showing an example of a display screen for multiple times of medical data on the researcher terminal 5 side. In addition, FIG. 29 illustrates an example of test items for Parkinson's disease, but the present invention is not limited thereto, and is similarly applied to test items for other types of diseases.

The screen includes a patient DID display field 19a, a view button 19b, a severity classification display field 19c, a diagnostic image display object 19d, an olfactory test display field 19e, a prescription information display field 19f, a blood pressure display field 19g, a pulse display field 19h, and a download button 19i. The patient DID display field 19a is a display field for displaying the patient DID. The view button 19b is a button for displaying answers to questions. In addition, since the view button 19b is the same as the view button 12e in FIG. 14, the description thereof will be omitted.

The severity classification display field 19c is a display field for displaying multiple times of severity classification information. In Parkinson's disease, the severity is classified into level I (symptoms only in one limb), level II (symptoms in both limbs), level III (postural reflex disorder is added), level IV (requires partial assistance in daily life), and level V (wheelchair dependent or bedridden). The severity classification information includes severity classification and diagnosis date and time of severity. As shown in the diagram, the severity classification information “I: 2012/06/08”, “II: 2017/10/31”, and “III: 2020/03/04” is displayed in the severity classification display field 19c. The diagnostic image display object 19d is a button for displaying multiple times of diagnostic images. In addition, the number of diagnostic image display objects 19d may be set based on the type of diagnostic image. As shown in the diagram, the diagnostic image display objects 19d for “MRI”, “DAT Scan”, “Cardiac MIBG Scintigraphy” and “Brain Perfusion SPECT” are provided.

The olfactory test display field 19e is a display field for displaying the results of multiple olfactory tests. The prescription information display field 19f is a display field for displaying multiple times of prescription information. The blood pressure display field 19g is a display field for displaying the results of multiple blood pressure tests. The pulse display field 19h is a display field for displaying the results of multiple pulse tests. The download button 19i is a button for downloading medical data.

The researcher terminal 5 transmits the patient DID and the researcher DID to the server 1. The server 1 receives the patient DID and the researcher DID transmitted from the researcher terminal 5, and acquires multiple times of medical data of the patient based on the received patient DID and researcher DID. Specifically, the server 1 acquires answers to all questions for each question type from the question DB 173 based on the patient DID. The server 1 extracts the medical data ID of the medical data, which is a transaction target, from the transaction history DB 176 based on the patient DID and the researcher DID. The server 1 acquires all pieces of the corresponding medical data from the medical data DB 174 based on the extracted medical data ID.

The server 1 transmits the acquired multiple times of medical data to the researcher terminal 5. The researcher terminal 5 integrates the multiple times of medical data transmitted from the server 1. The researcher terminal 5 displays the integrated multiple times of medical data on the screen. Specifically, the researcher terminal 5 extracts multiple times of severity classifications from the medical data in reverse chronological order of diagnosis date and time, and displays the extracted multiple times of severity classifications in the severity classification display field 19c.

The researcher terminal 5 extracts multiple times of olfactory test results from the medical data in chronological order of diagnosis date and time, and generates a graph showing the score of the olfactory test based on the extracted multiple times of olfactory test results. The researcher terminal 5 displays the generated graph in the olfactory test display field 19e. As shown in the diagram, a graph showing the score of the patient's olfactory test using OSIT-J (Odor Stick Identification Test for Japanese) is displayed in the olfactory test display field 19e. The horizontal axis indicates the number of tests, such as the first time, the second time, and so on, and the vertical axis indicates the score of the olfactory test. In addition, the horizontal axis may indicate the date of the olfactory test. In addition, when a plurality of olfactory test methods are used, the researcher terminal 5 may generate graphs corresponding to the respective test methods and display the graphs side by side in the olfactory test display field 19e.

The researcher terminal 5 extracts multiple times of prescription information from the medical data in reverse chronological order of diagnosis date and time, and displays the extracted multiple times of prescription information in the prescription information display field 19f. The researcher terminal 5 extracts multiple times of blood pressure (hypertension and hypotension) test results from the medical data in chronological order of diagnosis date and time, and generates a graph showing blood pressure values based on the extracted multiple times of blood pressure test results. The researcher terminal 5 displays the generated graph in the blood pressure display field 19g. As shown in the diagram, the horizontal axis indicates the number of tests, such as the first time, the second time, and so on, and the vertical axis indicates the value of blood pressure. In addition, the horizontal axis may indicate the date of the blood pressure test.

The researcher terminal 5 extracts multiple times of pulse test results from the medical data in chronological order of diagnosis date and time, and generates a graph showing the pulse rate based on the extracted multiple times of pulse test results. The researcher terminal 5 displays the generated graph in the pulse display field 19h. As shown in the diagram, the horizontal axis indicates the number of tests, such as the first time, the second time, and so on, and the vertical axis indicates the pulse rate. In addition, the horizontal axis may indicate the date of the pulse test.

When a click (touch) operation on the view button 19b is received, the researcher terminal 5 displays multiple times of answers to the corresponding question on the sub-screen. For example, when a click operation on the view button 19b corresponding to “PDQ-39” is received, the researcher terminal 5 generates a sub-screen for displaying the answer to the question. The researcher terminal 5 extracts multiple times of answers to “PDQ-39” from the received medical data, and displays the extracted multiple times of answers on the sub-screen.

When a click operation on the diagnostic image display object 19d is received, the researcher terminal 5 displays corresponding multiple times of diagnostic images on the sub-screen. For example, when a click operation on the diagnostic image display object 19d corresponding to “DAT Scan” is received, the researcher terminal 5 generates a sub-screen for displaying the diagnostic image. The researcher terminal 5 extracts multiple times of diagnostic images for “DAT Scan” from the received medical data, and displays the extracted multiple times of diagnostic images on the sub-screen. In addition, both the diagnostic image and the date and time when the diagnostic image was captured may be displayed on the sub-screen.

When a click operation on the download button 19i is received, the researcher terminal 5 writes medical data integrated based on multiple times of medical data in an Excel (registered trademark) file, a PDF (Portable Document Format) file, or the like. The researcher terminal 5 downloads the file in which the medical data is written to a designated folder.

In addition, FIG. 29 illustrates a display example of multiple times of medical data, but the present invention is not limited thereto, and is similarly applied to one time of medical data.

According to the present embodiment, by outputting multiple times of medical data in an integrated manner, the medical data can be used for long-term follow-up observation, determination of treatment effects, and the like. This can be useful for medical research.

### Fifth Embodiment

A fifth embodiment relates to a form in which patient's medical data is re-disclosed in a second period after a predetermined period. In addition, the description of the same contents as in the first to fourth embodiments will be omitted.

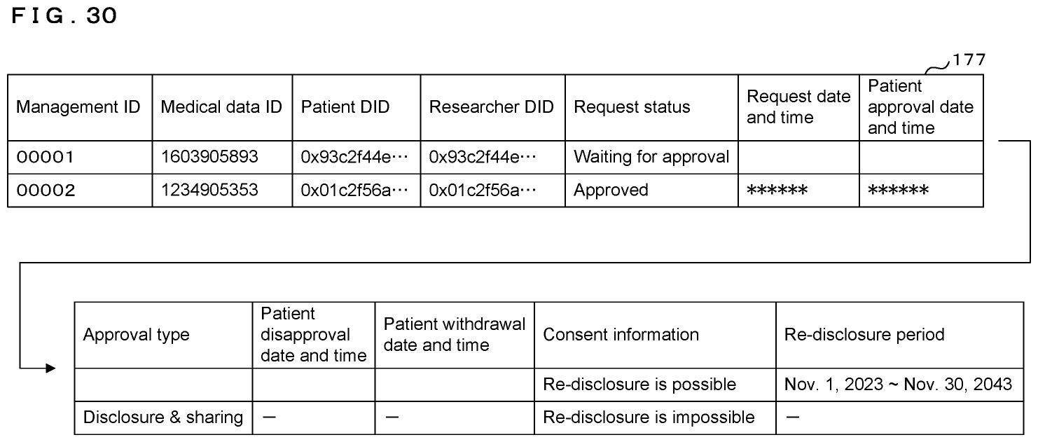

FIG. 30 is an explanatory diagram showing an example of the record layout of the management DB 177 in the fifth embodiment. In addition, the same contents as in FIG. 6 are denoted by the same reference numerals, and the description thereof will be omitted. The management DB 177 includes a consent information column and a re-disclosure period column.

The consent information column stores consent information regarding the re-disclosure of patient's medical data (for example, whether re-disclosure is possible or re-disclosure is not possible). The re-disclosure period column stores the re-disclosure period of medical data. The re-disclosure period is the second period after the predetermined period. For example, the predetermined period may be two years from the starting date, and the second period may be twenty years after two years. The starting date may be, for example, the registration date when the medical data is first registered in the node 21 of the blockchain 2, or may be the approval date when the patient's first approval for the request for disclosure of the medical data is obtained. In addition, the second period may be stored in advance in the re-disclosure period column, or may be set by the user.

When consent information regarding the re-disclosure of the medical data is received in the second period after the predetermined period, the patient terminal 4 transmits the received consent information to the server 1. When consent information for the medical data is obtained, the medical data can be re-disclosed in the second period. The server 1 stores the consent information transmitted from the patient terminal 4 in the management DB 177 in association with the medical data ID.

In the second period, when a medical data re-disclosure request is received from a researcher, the researcher terminal 5 transmits the received re-disclosure request to the server 1 in association with the researcher DID and the medical data ID. The server 1 receives the researcher DID, the medical data ID, and the re-disclosure request transmitted from the researcher terminal 5. The server 1 determines whether or not the medical data can be re-disclosed according to the received re-disclosure request.

Specifically, the server 1 acquires consent information and a second period (re-disclosure period) corresponding to the medical data from the management DB 177 based on the medical data ID. The server 1 determines whether or not the medical data can be re-disclosed based on the acquired consent information and second period. For example, in the second period, if the consent information is “re-disclosure possible”, the control unit 11 determines that the medical data can be re-disclosed. Alternatively, in the second period, if the consent information is “re-disclosure not possible”, the control unit 11 determines that the medical data cannot be re-disclosed.

When it is determined that the medical data can be re-disclosed, the server 1 transmits the re-disclosure request, the researcher DID, and the medical data ID transmitted from the server 1 to the patient terminal 4 of the patient who provided the medical data. The patient terminal 4 displays the re-disclosure request, the researcher DID, and the medical data ID transmitted from the server 1 on the screen. In addition, a history of past disclosure requests (researcher DID, disclosure request date and time, approval date and time, and the like) and the researcher's message or profile may be transmitted to the patient terminal 4 together with the re-disclosure request. In this case, the patient terminal 4 displays the history of past disclosure requests and the researcher's message or profile on the screen together with the re-disclosure request.

When the patient's re-approval information for the medical data re-disclosure request is received, the patient terminal 4 transmits the received re-approval information to the server 1. The re-approval information includes information indicating whether or not the medical data can be re-disclosed and the approval type (“disclosure only” or “disclosure & sharing”) for the medical data. The server 1 receives the re-approval information transmitted from the patient terminal 4. The server 1 determines whether or not the medical data can be re-disclosed according to the received re-approval information.

When it is determined that the medical data can be re-disclosed, the server 1 pays a predetermined token from the researcher to the patient through the node 21 of the blockchain 2. In addition, since the payment processing is the same as in the first embodiment, the description thereof will be omitted. In addition, if there is a service such as providing the medical data free of charge by the patient, the token payment processing may be omitted.

After performing the token payment processing, the node 21 of the blockchain 2 acquires the hash value of the medical data based on the medical data ID. The node 21 transmits the acquired hash value of the medical data to the server 1. The server 1 receives the hash value of the medical data transmitted from the node 21. The server 1 compares the received hash value of the medical data with the hash value of the medical data stored in the medical data DB 174.

When the hash values match each other, the server 1 acquires medical data corresponding to the hash value of the medical data from the medical data DB 174. The server 1 transmits the acquired medical data to the researcher terminal 5. The researcher terminal 5 receives the medical data transmitted from the server 1 and displays the medical data on the screen.

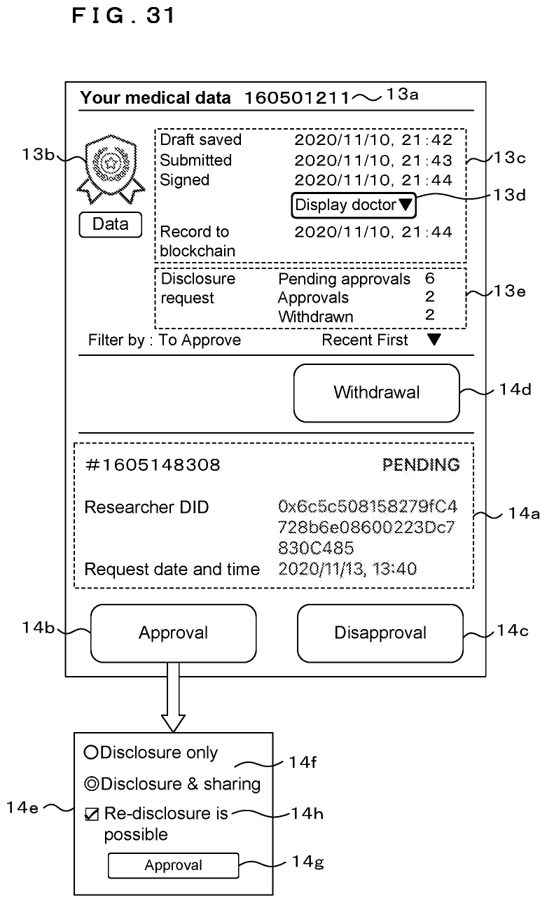

FIG. 31 is an explanatory diagram showing an example of a screen for receiving consent information. In addition, the same contents as in FIG. 18 are denoted by the same reference numerals, and the description thereof will be omitted.

The approval type setting dialog 14e includes a consent check box 14h. The consent check box 14h is a check box for receiving consent information regarding the re-disclosure of the patient's medical data in the second period (for example, 20 years) after the predetermined period (for example, 2 years). When a check operation on the consent check box 14h is received, the patient terminal 4 acquires the received consent information (for example, re-disclosure possible or re-disclosure not possible). In addition, the second period may be set by the user (for example, the patient).

When a touch operation on the intra-dialog approval button 14g is received, the patient terminal 4 transmits the approval type set by the approval type setting radio button 14f and the consent information acquired by the consent check box 14h to the server 1 in association with the medical data ID. The server 1 stores the approval type and the consent information transmitted from the patient terminal 4 in the management DB 177 in association with the medical data ID.

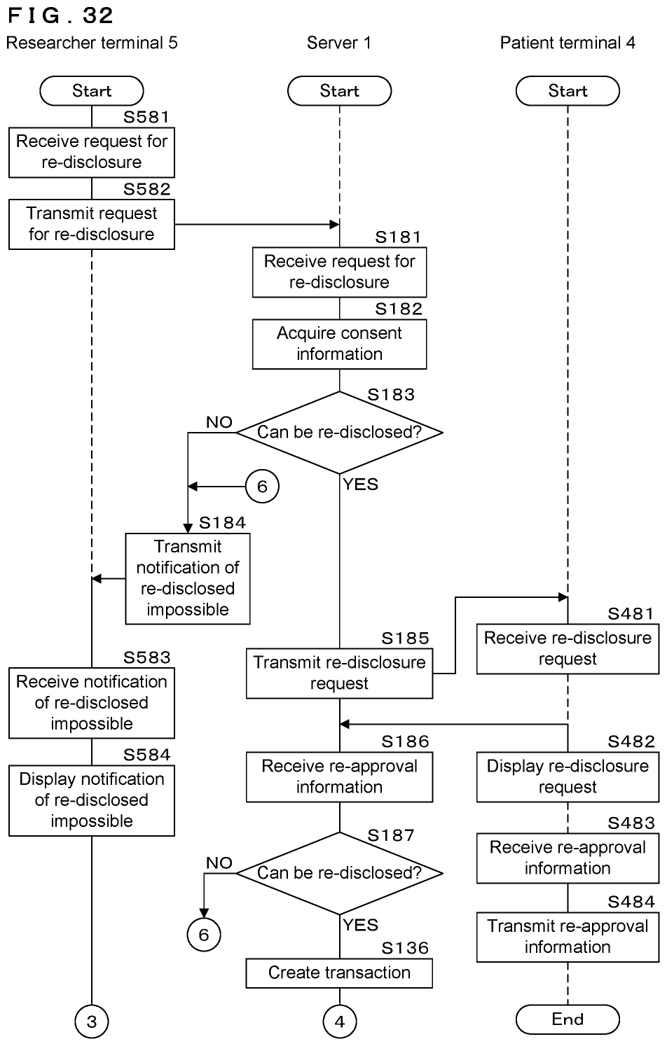

FIG. 32 is a flowchart showing a processing procedure when outputting medical data re-approved by the patient to the researcher terminal 5. In addition, the same contents as in FIGS. 25 and 26 are denoted by the same reference numerals, and the description thereof will be omitted.

The control unit 51 of the researcher terminal 5 receives a request for re-disclosure of medical data recorded in the storage unit 212 of the node 21 of the blockchain 2 through the input unit 54 (step S581). The control unit 51 transmits the received re-disclosure request to the server 1 through the communication unit 53 in association with the researcher DID and the medical data ID (step S582). The control unit 11 of the server 1 receives the researcher DID, the medical data ID, and the re-disclosure request transmitted from the researcher terminal 5 through the communication unit 13 (step S181).

Based on the received medical data ID, the control unit 11 acquires the second period and the consent information corresponding to the medical data from the management DB 177 of the mass storage unit 17 (step S182). The control unit 11 determines whether or not the medical data can be re-disclosed based on the acquired consent information and second period (step S183).

When it is determined that the medical data cannot be re-disclosed (NO in step S183), the control unit 11 transmits a notification indicating that the medical data cannot be re-disclosed to the researcher terminal 5 through the communication unit 13 (step S184). The control unit 51 of the researcher terminal 5 receives the notification of re-disclosure impossible transmitted from the server 1 through the communication unit 53 (step S583), and displays the received notification of re-disclosure impossible on the display unit 55 (step S584).

When the control unit 11 determines that the medical data can be re-disclosed (YES in step S183), the control unit 11 transmits the received researcher ID, medical data ID, and re-disclosure request to the patient terminal 4 of the patient who provided the medical data (step S185). The control unit 41 of the patient terminal 4 receives the researcher ID, medical data ID, and re-disclosure request transmitted from the server 1 through the communication unit 43 (step S481). The control unit 41 displays the received researcher ID, medical data ID, and re-disclosure request on the display unit 45 (step S482).

In response to the received re-disclosure request, the control unit 41 receives patient's re-approval information for the medical data (step S483), and transmits the received re-approval information to the server 1 through the communication unit 43 (step S484). The re-approval information includes information indicating whether or not the medical data can be re-disclosed and the approval type for the medical data. The control unit 11 of the server 1 receives the re-approval information transmitted from the patient terminal 4 through the communication unit 13 (step S186).

Based on the received re-approval information, the control unit 11 determines whether or not the medical data can be re-disclosed (step S187). When it is determined that the medical data cannot be re-disclosed (NO in step S187), the control unit 11 returns to the processing of step S184. When it is determined that the medical data can be re-disclosed (YES in step S187), the control unit 11 performs the processing of step S136.

According to the present embodiment, it is possible to re-disclose the medical data in the second period after the predetermined period based on the consent information regarding the re-disclosure of the patient's medical data.

According to the present embodiment, re-disclosure of medical data makes it possible to promote progress in medical technology by utilizing the medical data.

It should be considered that the embodiments disclosed are examples in all points and not restrictive. The scope of the present invention is defined by the claims rather than the meanings set forth above, and is intended to include all modifications within the scope and meaning equivalent to the claims.

It is to be noted that, as used herein and in the appended claims, the singular forms “a”, “an”, and “the” include plural referents unless the context clearly dictates otherwise.

It is to be noted that the disclosed embodiment is illustrative and not restrictive in all aspects. The scope of the present invention is defined by the appended claims rather than by the description preceding them, and all changes that fall within metes and bounds of the claims, or equivalence of such metes and bounds thereof are therefore intended to be embraced by the claims.

What is claimed is:

1-20. (canceled)

21. An information processing method, comprising:
acquiring a patient ID for identifying a patient, the patient's attributes, and the patient's answers to medical questions;
acquiring a doctor ID for identifying a doctor by causing a patient terminal device of the patient to read the doctor ID;
transmitting the patient ID, the attributes, and the answers of the patient to a doctor terminal device corresponding to the acquired doctor ID;
receiving an input of medical examination data for the patient by the doctor through the doctor terminal device on which the patient ID, the attributes, and the answers of the patient are displayed;
receiving a request to transmit medical data including the patient's attributes, the answers and the input medical examination data;
outputting a hash value of the medical data to a blockchain system;
receiving a request for disclosure of the medical data recorded in the blockchain system through a researcher terminal device of a researcher; and
outputting the medical data to the researcher terminal device when the patient's approval for the disclosure request from the researcher is obtained.

22. The information processing method according to claim 21,
wherein medical data with an electronic signature electronically signed by using a private key of the doctor is acquired from the doctor terminal device, and
the acquired medical data is output to the patient terminal device.

23. The information processing method according to claim 21,
wherein, when an instruction to output the medical data to the blockchain system is received from the patient terminal device, the hash value of the medical data is output to the blockchain system.

24. The information processing method according to claim 21,
wherein the doctor ID is acquired by causing the patient terminal device to read a two-dimensional code describing the doctor ID.

25. The information processing method according to claim 21,
wherein, when the patient's approval for the disclosure request is obtained, a predetermined token is paid from the researcher's wallet address to the patient's wallet address through the blockchain system.

26. The information processing method according to claim 21,
wherein approval information including an approval for the medical data disclosure request or the approval and a second approval of the patient for a sharing request to share the medical data, for which the approval for the medical data disclosure request has been obtained, with other researchers is received from the patient terminal device, and
the received approval information is stored in association with the medical data.

27. The information processing method according to claim 21,
wherein consent information regarding re-disclosure of the medical data is received from the patient terminal device in a second period after a predetermined period,
when a request for re-disclosure of medical data for which the consent information has been obtained is received through the researcher terminal device of the researcher in the second period, the received re-disclosure request is transmitted to the patient terminal device, and
when the patient's re-approval for the re-disclosure request is obtained, the medical data is output to the researcher terminal device.

28. The information processing method according to claim 21,
wherein information regarding results of research conducted by using the medical data is received from the researcher terminal device, and
the received information regarding the research results is output to a patient terminal device of a patient who provided the medical data.

29. The information processing method according to claim 22,
wherein a transaction history including a medical data ID for identifying the medical data, a transaction date and time of the medical data, the patient ID, a researcher ID for identifying the researcher, and a hash value of the medical data is acquired, and
the acquired transaction history is transmitted to the researcher terminal device, the doctor terminal device, or the patient terminal device.

30. The information processing method according to claim 22,
wherein the patient ID, information regarding the disclosure request, and a date and time of the doctor's signature for the medical data are acquired, and
the acquired patient ID, the acquired information regarding the disclosure request, and the acquired signature date and time for the medical data are transmitted to the researcher terminal device.

31. The information processing method according to claim 21,
wherein, when a request for disclosure of the medical data is received from the researcher terminal device, the received disclosure request is transmitted to a patient terminal device of a patient who provided the medical data.

32. The information processing method according to claim 21,
wherein the medical data ID, information regarding the disclosure request, and the doctor ID are acquired, and
the acquired medical data ID, the acquired information regarding the disclosure request, and the acquired doctor ID are transmitted to the patient terminal device.

33. The information processing method according to claim 22,
wherein a status of the medical data indicating whether the medical data is stored but an electronic signature from the doctor has not been acquired, an electronic signature for the medical data has been acquired from the doctor, or the medical data has been recorded in the blockchain system is transmitted to the patient terminal device, and
an object indicating the status of the medical data is displayed on the patient terminal device.

34. An information processing apparatus, comprising:
one or more processing devices; and
one or more storage devices storing instructions for causing the one or more processing devices executing the following processing of:
acquiring a patient ID for identifying a patient, the patient's attributes, and the patient's answers to medical questions;
acquiring a doctor ID for identifying a doctor by causing a patient terminal device of the patient to read the doctor ID;
transmitting the patient ID, the attributes, and the answers of the patient to a doctor terminal device corresponding to the acquired doctor ID;
receiving an input of medical examination data for the patient by the doctor through the doctor terminal device on which the patient ID, the attributes, and the answers of the patient are displayed;
receiving a request to transmit medical data including the patient's attributes and answers and the input medical examination data;
outputting a hash value of the medical data to a blockchain system;
receiving a request for disclosure of the medical data recorded in the blockchain system through a researcher terminal device of a researcher; and
outputting the medical data to the researcher terminal device when the patient's approval for the disclosure request from the researcher is obtained.

35. A non-transitory computer-readable storage medium storing a program that causes a computer to execute processes of:
acquiring a patient ID for identifying a patient, the patient's attributes, and the patient's answers to medical questions;
acquiring a doctor ID for identifying a doctor by causing a patient terminal device of the patient to read the doctor ID;
transmitting the patient ID, the attributes, and the answers of the patient to a doctor terminal device corresponding to the acquired doctor ID;
receiving an input of medical examination data for the patient by the doctor through the doctor terminal device on which the patient ID, the attributes, and the answers of the patient are displayed;
receiving a request to transmit medical data including the patient's attributes and answers and the input medical examination data;
outputting a hash value of the medical data to a blockchain system;
receiving a request for disclosure of the medical data recorded in the blockchain system through a researcher terminal device of a researcher; and
outputting the medical data to the researcher terminal device when the patient's approval for the disclosure request from the researcher is obtained.

36. A non-transitory computer-readable storage medium storing a program that causes a computer to execute processes of:
acquiring a patient ID for identifying a patient;
receiving the patient's attributes and the patient's answers to medical questions;
acquiring a doctor ID for identifying a doctor;
transmitting the patient ID, the attributes, and the answers of the patient in association with the acquired doctor ID;
receiving medical data including the patient's attributes and answers and the doctor's medical examination data for the patient; and
transmitting information indicating whether or not the patient's approval for a disclosure request from a researcher has been obtained when the researcher's disclosure request for the medical data recorded in the blockchain system is received.

37. The program according to claim 36 causing the computer to execute processes of:
receiving an instruction to output the medical data to the blockchain system; and
transmitting the received instruction for output to the blockchain system.

38. The program according to claim 36 causing the computer to execute processes of:
reading a two-dimensional code describing the doctor ID; and
acquiring the doctor ID from the read two-dimensional code.

39. The program according to claim 36 causing the computer to execute processes of:
receiving approval information including an approval for the medical data disclosure request or the approval and a second approval of the patient for a sharing request to share the medical data, for which the approval for the medical data disclosure request has been obtained, with other researchers; and
transmitting the received approval information in association with a medical data ID for identifying the medical data.

40. The program according to claim 36 causing the computer to execute processes of:
receiving a status of the medical data indicating whether the medical data is stored but an electronic signature from the doctor has not been acquired, an electronic signature for the medical data has been acquired from the doctor, or the medical data has been recorded in the blockchain system; and
displaying an object indicating the received status of the medical data.
